(19) United States
(12) Patent Application Publication
(10) Pub. No.: US 2024/0209018 A1
(43) Pub. Date: Jun. 27, 2024

(54) UNIVERSAL LINKER REAGENTS FOR DNA SYNTHESIS

(71) Applicants: GENSCRIPT USA INC., Piscataway, NJ (US); CustomArray, Inc., Redmond, WA (US)

(72) Inventors: Michael W. REED, Piscataway, NJ (US); Cheng-Hsien WU, Piscataway, NJ (US); John COOPER, Piscataway, NJ (US); Robert O. DEMPCY, Piscataway, NJ (US)

(21) Appl. No.: 18/551,834

(22) PCT Filed: Mar. 22, 2022
(86) PCT No.: PCT/US2022/021287

§ 371 Date: Sep. 21, 2023

Related U.S. Application Data
(60) Provisional application No. 63/164,363, filed on Mar. 22, 2021.

(51) Int. Cl. C07H 21/04; C07C 247/04; C07C 275/10; C07D 491/18; C07F 9/24; C25B 3/05; C25B 3/25; C25B 3/29

(52) U.S. Cl. CPC C07H 21/04; C07C 247/04; C07C 275/10; C07D 491/18; C07F 9/2458; C25B 3/05; C25B 3/25; C25B 3/29

(57) ABSTRACT

Provided herein are methods and compositions for oligonucleotide synthesis utilizing universal linker phosphoramidites. Methods and reagents are described with DNA synthesis using controlled pore glass (CPG) solid supports, and on platinum coated electrodes for electrochemical DNA synthesis. The universal linkers can be used as spacers in single-column PCR primer synthesis to generate 2 strands with free 3′-hydroxy termini after cleavage. The methods and compositions utilize a solid support system for synthesis of oligonucleotides, wherein the support has platinum electrodes and a universal linker, optionally wherein the platinum electrode is coated with an amine. The methods and compositions further describe use of universal linker phosphoramidites and the platinum electrode is coated with a monosaccharide, or a disaccharide.

### CROSS-REFERENCE TO RELATED APPLICATION

This is an International Application under the Patent Cooperation Treaty, claiming priority to U.S. Provisional Patent Application No. 63/164,363, filed Mar. 22, 2021 the contents of which are incorporated herein by reference in their entirety.

### TECHNICAL FIELD OF THE INVENTION

The present invention provides an electrode array for electrochemical synthesis of oligomers by means of universal linker technology in combination with a solid support device having coated electrodes, e.g., platinum electrodes.

### BACKGROUND OF THE INVENTION

Rapid developments in the field of DNA microarrays have led to a number of methods for synthetic preparation of DNA. Such methods include spotting pre-synthesized oligonucleotides, photolithography using mask or maskless techniques, in situ synthesis by printing reagents, and in situ parallel synthesis on a microarray of electrodes using electrochemical deblocking of protective groups. A review of oligonucleotide microarray synthesis is provided by, for example, Gao et al., Biopolymers 2004, 73:579. The synthetic preparation of a peptide array was reported in year 1991 using photo-masking techniques. This method was extended in year 2000 to include an addressable masking technique using photogenerated acids and/or in combination with photosensitizers for deblocking. Reviews of peptide microarray synthesis using photolabile deblocking are provided by: Pellois et al., J. Comb. Chem. 2000, 2:355 and Fodor et al., Science, 1991, 251:767. Spotting pre-synthesized peptides or isolated proteins has been used to create peptide arrays. A review of protein or peptide arrays is provided by: Cahill and Nordhoff, Adv. Biochem. Engin/Biotechnol. 2003, 83:177. Universal Supports may be used in DNA synthesis due to the labor intensiveness in using four different solid supports (or more) to prepare 3′-unmodifed oligonucleotides.

Two different types of Universal Linkers (UL) are commercially available. Both types of linkers have vicinal diol structures which are individually protected as acid sensitive 4,4′-dimethoxytrityl (DMT) groups (for oligo extension) and base sensitive acyl groups (for release of oligos during deprotection). The first type of linker (Universal Support III, USIII) is released by treatment with anhydrous ammonia (Azhayev, 2001, Nucleosides Nucleotides Nucleic Acids, 20(4-7):539-50; Yagodkin, 2011, Nucleosides Nucleotides Nucleic Acids, 30(7-8):475-89) and is described in, for example, U.S. Pat. No. 6,770,754. The second type of UL (UNYLINKER™ or UNYSUPPORT™) is released with aqueous ammonia (Guzaev, 2003) or anhydrous methylamine gas (U.S. Pat. No. 7,202,264). Electrochemical parallel DNA synthesis on CMOS (complementary metal oxide semiconductor) type electrode arrays have been described (Maurer et. al, 2006, PLoS One. 2006 Dec. 20; 1(1):e34; U.S. Pat. No. 10,525,436). In this application, the CMOS chip surface is coated before DNA synthesis with an absorbed porous reaction layer over each platinum electrode. DNA synthesis starts from hydroxyl groups on a porous layer. After DNA synthesis, the oligo is either left on the chip or the oligo is cleaved from the chip surface. One problem with the absorbed porous coating is the long cleavage time for release of the oligo. The long cleavage time slows DNA synthesis production and may affect DNA quality. The released oligos may still have an absorbed coated molecule linked to the 3′-terminus. 3′-modification of the DNA strand is a problem for some applications (such as PCR primers) since 3′-modification blocks polymerase extension. Another problem with the absorbed coating on electrodes is degradation during DNA synthesis or when used for multiple hybridization assays.

The present invention addresses these challenges by providing methods for synthesis of oligonucleotides utilizing improved solid support media having universal linkers.

### BRIEF SUMMARY OF THE INVENTION

Provided herein are methods and compositions for oligonucleotide synthesis utilizing universal linkers on coated electrodes or as spacers in single-column PCR primer synthesis.

In one embodiment, the methods and compositions may comprise a solid support system for synthesis of oligonucleotides, wherein the support comprises platinum electrodes and a universal linker. In various embodiments, the platinum electrode is first coated with a substituted or unsubstituted aliphatic group, a substituted or unsubstituted aliphatic ether, a substituted or unsubstituted heteroalkyl group, a substituted or unsubstituted aromatic group, e.g., polybenzylalcohol group, a substituted or unsubstituted heteroaryl group, a substituted or unsubstituted heterocyclic group, to allow DNA synthesis to initiate. The alcohol coated electrodes are further functionalized with cleavable Universal linkers, to allow release of DNA after synthesis and deprotection. In some embodiments of the present disclosure, solid supports may include in coated surfaces platinum electrodes and dielectrics (insulators) that separate platinum electrodes. Dielectric may be at least one selected from the group consisting of silicon oxynitride, silicon nitride, silicon dioxide, and tetraethyl orthosilicate (TEOS).

In an embodiment, methods for synthesis of oligonucleotides may comprise using the compositions described herein.

In an aspect, the present disclosure may be related to a solid support system for synthesis of oligonucleotides, in which the support may include a planar surface and a universal linker, in which the universal linker may be coupled (attached or connected) to the planar surface.

In another aspect, the planar surface may be coated with an amine prior to attaching the universal linker.

In another aspect, the planar surface may be coated with carboxylic acid. In another aspect, the planar surface may be silicon, titanium, or platinum.

In another aspect, the solid support system may contain Formula (I), (III), or (IV), optionally, in which the universal linker may be coupled to the planar surface by reacting the planar surface with a compound of Formula (II), (V), (VI), (VII), (VIII), (IX), (X), or combinations thereof,

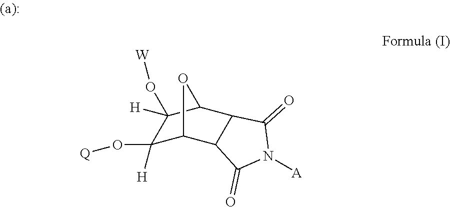

wherein, when A is a linking moiety attached to a coated platinum electrode comprising a substituted or unsubstituted aliphatic group, a substituted or unsubstituted aliphatic ether, a substituted or unsubstituted heteroalkyl group, a substituted or unsubstituted aromatic group, a substituted or unsubstituted heterocyclic group, one of W or Q is a blocking group that is cleavable under basic or neutral conditions, while the other of W or Q is H, or a blocking group that is cleavable under acidic conditions; or
wherein, when A is H, a substituted or unsubstituted aliphatic group, a substituted or unsubstituted aliphatic ether, a substituted or unsubstituted heteroalkyl group, a substituted or unsubstituted aromatic group, a substituted or unsubstituted heterocyclic group, one of Q or W is a linker moiety attached to a coated platinum electrode that is cleavable under basic or neutral conditions, while the other of W or Q is H, or a blocking group that is cleavable under acidic conditions;

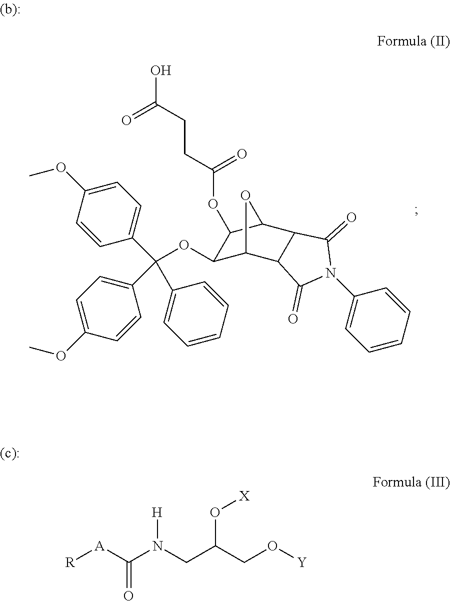

wherein
R is alkyl, aryl, heteroalkyl or heteroaryl attached to platinum electrode or other base material;
A is NH, O, S, alkyl, or aryl;
X is acyl, aroyl, or silyl; and
Y is dimethoxytrityl group or a protecting group removable under acidic or neutral conditions;

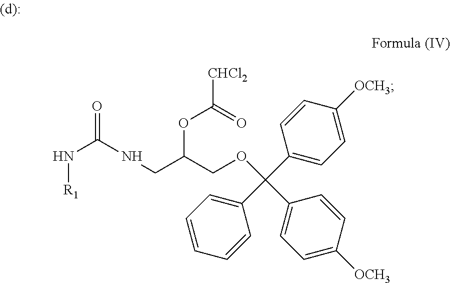

R₁ is alkyl, aryl, heteroalkyl, or heteroaryl attached to platinum electrode or other base material

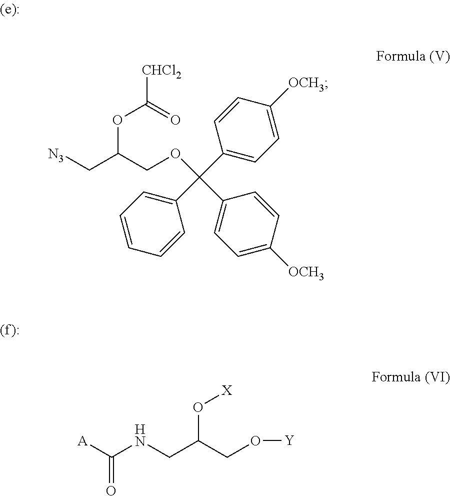

wherein
A is

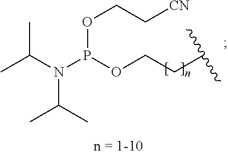

X is acyl, aroyl, or silyl; and
Y is dimethoxytrityl group or a protecting group removable under acidic or neutral conditions;

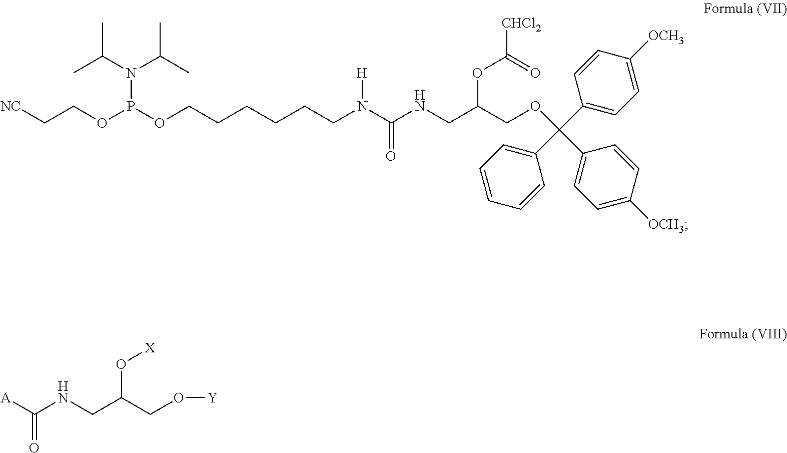

wherein
A is

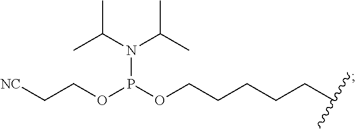 [SMILES: *CCCCCOP(OCCC#N)N(C(C)C)C(C)C]

X is acyl, aroyl, or silyl; and
Y is dimethoxytrityl group or a protecting group removable under acidic or neutral conditions;

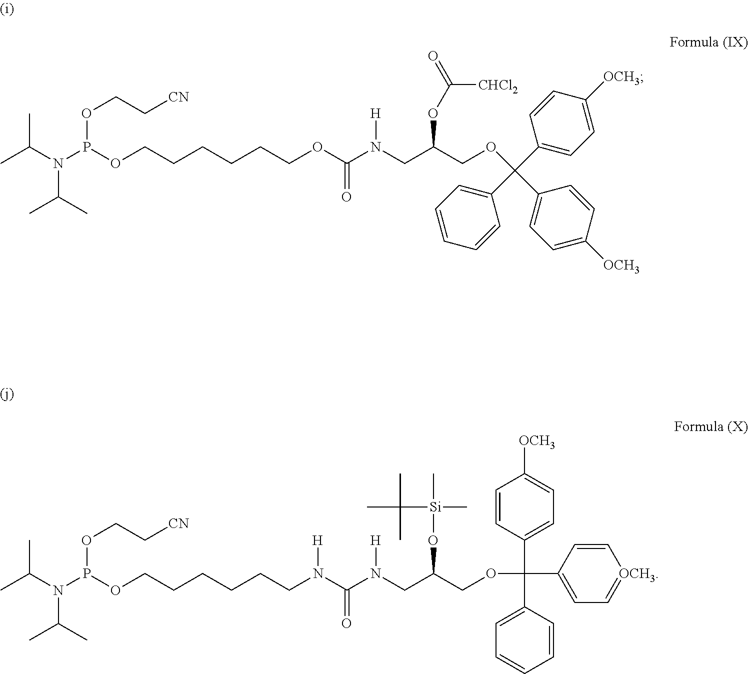

In another aspect, the compound may be of Formula (II), (IV), (V), (VII), (IX), (X), or combinations thereof.

In another aspect, the compound may be of Formula (VII), (IX), or (X).

In another aspect, the planar surface may be coated with a monosaccharide or a disaccharide.

In another aspect, the monosaccharide may be selected from the group consisting of allose, altrose, arabinose, deoxyribose, erythrose, fructose, galactose, glucose, gulose, idose, lyxose, mannose, psicose, L-rhamnose, ribose, ribulose, sedoheptulose, D-sorbitol, sorbose, sylulose, tagatose, talose, threose, xylulose, and xylose and the disaccharide is selected from the group consisting of sucrose, amylose, cellobiose, lactose, maltose, melibiose, palatinose, and trehalose.

In an aspect, the present disclosure may be related to a method for synthesis of oligonucleotides comprising: (a) providing an electrode device with a planar surface; (b) coupling the surface with a universal linker; and (c) synthesizing the oligonucleotide.

In another aspect, the method may further include a step of depositing carboxylic acid electrochemically reducing the carboxylic acid onto the planar surface.

In another aspect, the method may further include depositing an amine coating onto the activated carboxylic acid.

In another aspect, the planar surface may contain silicon, titanium, or platinum.

In another aspect, the solid support system may contain Formula (I), (III), or (IV), optionally, in which the universal linker may be coupled to the planar surface by reacting the planar surface with a compound of Formula (II), (V), (VI), (VII), (VIII), (IX). (X), or combinations thereof.

In an aspect, the present disclosure may be related to a method for synthesis of oligonucleotide primer pairs comprising providing a solid support comprising a first universal linker immobilized on a surface of the solid support, performing a first phosphoramidite DNA synthesis to generate a first oligonucleotide primer, wherein the 3′ end of the first oligonucleotide primer is attached to the first universal linker, coupling a second universal linker to the 5′ end of the first oligonucleotide primer, performing a second phosphoramidite DNA synthesis to generate a second oligonucleotide primer, wherein the 3′ end of the second oligonucleotide primer is attached to the second universal linker, and contacting the solid support with a releasing agent thereby releasing the first and the second oligonucleotide primers from the solid support, wherein each of the released first oligonucleotide primer and the released second oligonucleotide primer contains a 3′-hydroxy group.

In another aspect, the first universal linker may be immobilized to the solid support by reacting the solid support with a first compound of Formula (VI), (VII), (VIII), (IX), (X), or combinations thereof.

In another aspect, the first compound is of Formula (VII), (IX), or (X).

In another aspect, the second universal linker may be attached to the first oligonucleotide primer by reacting the first oligonucleotide primer with a second compound of Formula (VII), (IX), or (X).

In another aspect, the releasing agent may include 4M methylamine/MeOH or TEA:3HF.

In another aspect, the method may further include removing protecting groups from the released first oligonucleotide primer and the released second oligonucleotide primer with AMA (1:1, 37% ammonium hydroxide:40% methylamine).

In another aspect, the released first oligonucleotide primer and the released second oligonucleotide primer may be about the same length.

In another aspect, the concentration ratio of the released first oligonucleotide primer and the released second oligonucleotide primer may be about 1:1.

In another aspect, the method may be performed in a single column.

In another aspect, the compound may be of Formula (X).

In an aspect, the present disclosure may be related to a compound of Formula (XI),

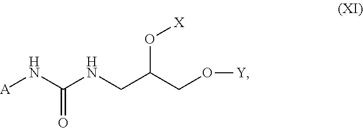

wherein
A is

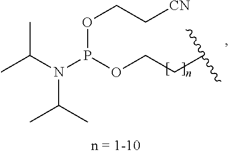

X is acyl, aroyl, or silyl, and
Y is dimethoxytrityl group or a protecting group removable under acidic or neutral conditions.

In another aspect, n may be 5.

In another aspect, X may be silyl.

In another aspect, the silyl may be trimethylsilyl, Triethylsilyl, tert-butyldiphenylsilyl, tert-butyldimethylsilyl, or triisopropylsilyl.

In another aspect, the silyl may be tert-butyldimethylsilyl.

In another aspect, Y may be dimethoxytrityl group.

In another aspect, the compound may include (R)-5-((bis(4-methoxyphenyl)(phenyl)methoxy)methyl)-2,2,3,3-tetramethyl-8-oxo-4-oxa-7,9-diaza-3-silapentadecan-15-yl 2-cyanoethyl diisopropylphosphoramidite.

In an aspect, the present disclosure may be related to a method for synthesis of oligonucleotides including: (a) providing the solid support system of the present disclosure; (b) coupling the surface with a universal linker; and (c) synthesizing the oligonucleotide.

In another aspect, titanium may include titanium nitride.

In another aspect, the planar surface may include a plurality of platinum electrodes separated by at least one dielectric.

In another aspect, the at least one dielectric may be selected from the group consisting of silicon oxynitride, silicon nitride, silicon dioxide, and tetraethyl orthosilicate (TEOS).

### BRIEF DESCRIPTION OF THE DRAWINGS

FIG. 1 shows a comparison of Universal support structures. In each case, DNA Synthesis starts from dimethoxytrityl group (DMT) in linker structure. Treatment with base releases oligonucleotides with unmodified 3′-hydroxy terminus.

FIG. 2 demonstrates the release vs. immobilization of synthetic oligonucleotide. Treatment with anhydrous ammonia in methanol rapidly cleaves the chloroacetyl group, while treatment with fluoride ion cleaves a silyl protecting group. The resulting dephosphorylation releases oligo. Treatment with t-butylamine in ACN rapidly cleaves the cyanoethyl group and immobilizes the oligo to the surface.

FIG. 3 shows reaction of UNYLINKER™ Acid with amine coated solid supports. The UNYLINKER™ structure is “pre-organized” with the vicinal hydroxyl groups on the same side of a rigid ring system. When the base sensitive succinate linkage is hydrolyzed, dephosphorylation and release of 3′-hydroxy oligonucleotides is very rapid.

FIG. 4 shows a method for synthesis of Linker amine 3. The novel (R) isomer is easily prepared and used as a synthon for each Universal Linker Phosphoramidite (ULP).

FIG. 5 shows a method for synthesis of ULP 1. 4-nitrophenyl 6-(tert-butyldimethylsilyloxy)hexylcarbamate is first prepared and coupled to Linker amine 3 to give a urea bond. After dichloroacetylation, selective removal of the TBDMS group with tetrabutylammonium fluoride (TBAF) is followed by phosphitylation to give the phosphoramidite.

FIG. 6 shows a method for synthesis of ULP 2. Similar structure to ULP 1, but a carbamate bond is created by reaction of TBDPS protected 1,6-hexanediol with p-nitrophenyl chloroformate and coupling to Linker amine 3. After dichloroacetylation, selective removal of the TBDPS group with TBAF is followed by phosphitylation to give the phosphoramidite.

FIG. 7 shows a method for synthesis of ULP 3. Similar structure to ULP 1, but a silyl protecting group (TBDMS) was used instead of dichloroacetyl protecting group.

FIG. 8 shows a method for synthesis of DMT-CPG using a novel p-nitrophenyl (PNP) ester. p-nitrophenyl (PNP) ester converts amine coated surfaces to DMT protected alcohol surfaces. This method gives stable urea bond in 1 step (no EDC coupling). Aminohexanol provides additional long linker. Loading of DMT CPG can be accurately measured prior to DNA synthesis.

FIG. 9 shows use of DMT-CPG assay to evaluate coupling efficiency and cleavage efficiency of universal linkers.

FIG. 10 is a diagram that shows how 2 primers (forward and reverse) are used to bracket the dsDNA amplification region (amplicon). PCR primers must have unmodified 3′-ends or they will not be extended by Taq polymerase during the Polymerase Chain Reaction.

FIG. 11 represents simultaneous DNA synthesis of PCR primer pairs. DNA is synthesized as usual with Universal Linker phosphoramidite spacer between the Primer sequences. Deprotection gives 1:1 mix of Primers with unmodified 3′—OH. Primer 1 has residual 5′-UL fragment, which will not affect PCR performance.

FIG. 12 shows the cleavage kinetics of BHQ1 from CPG-UL3-BHQ1 in 1.6% (v/v) TEA:3HF/MeOH at 22 C (+/−0.2 C) as determined by spectrophotometric assay.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments and is not intended to be limiting. Instead, the scope of the present disclosure will be established by the appended claims.

Advantages of the present disclosure may include, for example, (1) improved oligonucleotide yields by using silyl groups to protect the secondary hydroxyl group of universal linkers, (2) single isomer at the secondary hydroxyl carbon allowing for greater ease in chemical analysis of the universal linker phosphoramidite since fewer isomeric forms of the phosphoramidite reagent may exist, (3) improved cleavage by using “pre-organized” vicinal syn oxygen functionalized groups in universal linkers, and (4) reduced labor by automatically synthesizing and purifying a 1:1 mixture of primer pairs in a single operation.

With the introduction of high throughput DNA synthesizers, the relevance of universal solid supports seems more important than ever. Conventional synthesis supports contain linkers that have the first base attached to the linkers. For example, four (A, T, G, C) solid supports are required for synthesis of oligodeoxyribonucleotides. In contrast, universal solid supports contain universal linkers that do not have the first base attached to them. As such, universal solid supports may permit the use of one support for all syntheses. Universal linkers can, therefore, (1) eliminate the need for an inventory of nucleoside-linker-supports. (2) minimize the possibility of error in the selection of the correct nucleoside-linker-support type. (3) reduce time and eliminate possible error in the generation of an array of nucleoside-linker-supports in high throughput synthesizers, and (4) allow for the preparation of oligonucleotides that contain a 3′—OH terminal for any selected nucleoside (A, T, G, C) when a given support may conventionally not offer this option.

Provided herein is an improved method of synthesis of 3′-unmodified oligonucleotides on DNA synthesis electrodes. The synthesis of 3′-unmodified oligos is greatly simplified with improvements in quality and the efficiency of cleavage of the synthesized oligonucleotides. Reagents for DNA synthesis described here contain a Universal Linker that can be applied to coated electrode surfaces.

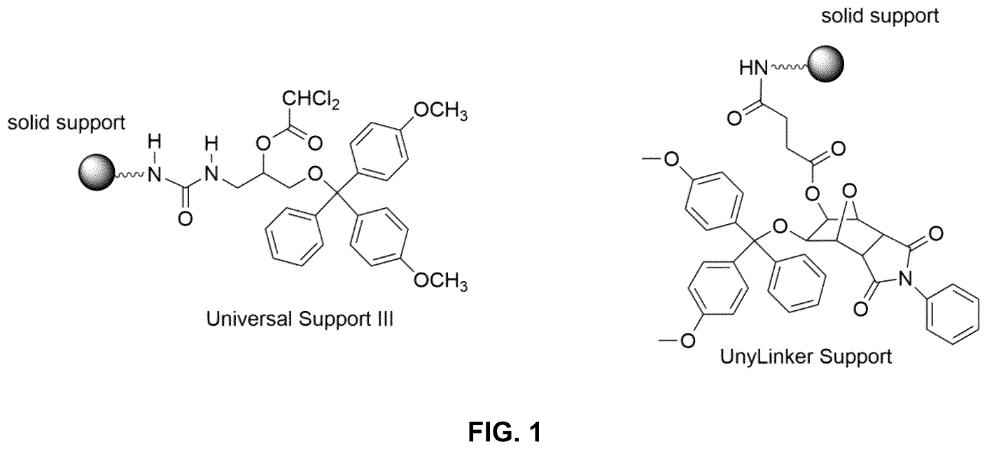

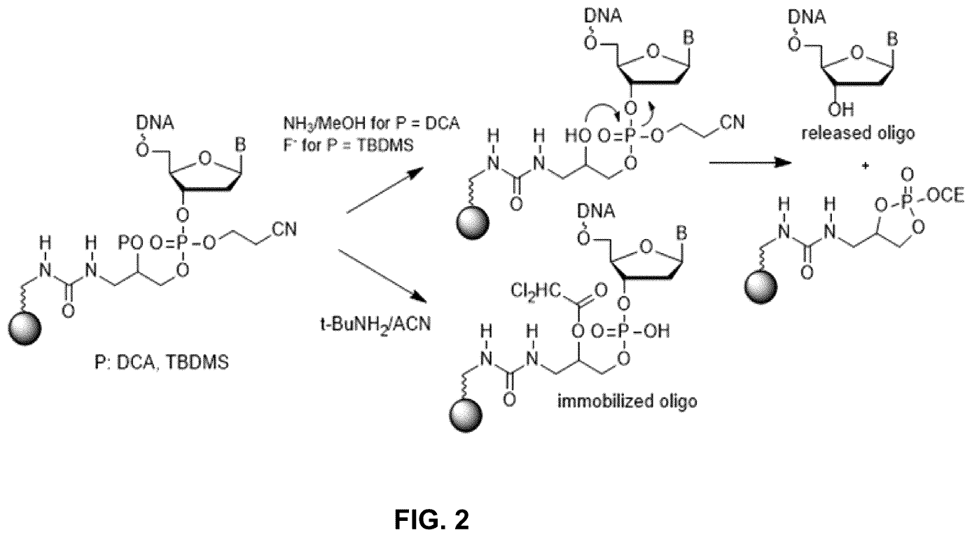

There are two different types of universal linkers (see, for example, FIG. 1) that have been commercialized and are sold by Glen Research (Sterling, VA). Both types of linkers have vicinal diol structures which are individually protected as acid sensitive DMT groups (for oligo extension) and base sensitive acyl groups (for release of oligo during deprotection). In each case, DNA synthesis starts from dimethoxytrityl group (DMT) in the linker structure. Treatment with base releases oligonucleotides with unmodified 3′-hydroxy terminus (see FIG. 2).

The compositions and methods comprise use of universal linker solid support structures comprising coated platinum electrodes wherein the coated surface is coupled to a universal linker. For linkers with structure of Universal Support III, deprotection of the dichloroacetyl group on the secondary hydroxyl with anhydrous ammonia in methanol releases 3′-unmodified nucleic acid strands for further deprotection and purification. The dichloroacetyl group is very reactive to base but aqueous ammonia deprotection also rapidly cleaves the cyanoethyl protecting groups from the phosphate and gives lower yield of released synthesized oligonucleotide from the electrode surface. The cyanoethyl groups can also be selectively removed with t-butylamine or DBU to immobilize the synthetic oligonucleotide strands to the solid support.

Although the dichloroacetyl protecting group on the secondary hydroxyl group has shown good performance in Universal Support III, competing hydrolysis of cyanoethyl group leads to low yields. For example, a “Universal Linker Phosphoramidite” has been described in the literature (see Yagodkin, 2009). They used a more stable 2,4-dichloroacetyl protecting group and showed 15-25% lower yield of released oligonucleotide than with dichloroacetyl protecting group. Herein we disclose that silyl groups (such as, e.g., TBDMS or TBDPS) can be used to protect the secondary hydroxyl group. This group can be removed with fluoride ion (such as, e.g., TBAF or TREAT HF), thus preventing competing hydrolysis of the cyanoethyl group and giving higher yields of released oligonucleotide.

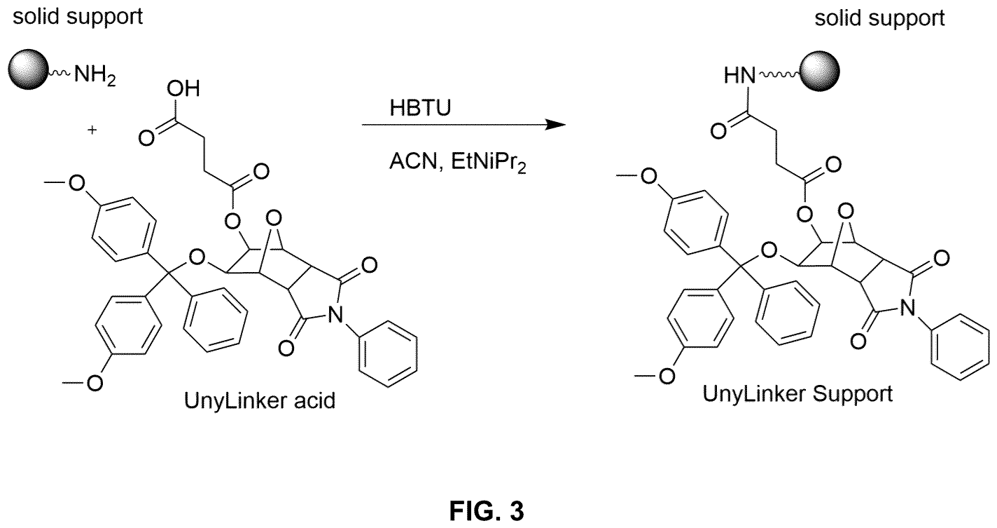

The universal linker may have a conformationally rigid and chemically stable bridge head ring oxygen atom carrying a 4,4′-dimethoxytrityl (DMT) and succinyl groups locked in a syn orientation (Ravikumar et al., Org. Process Res. Dev. 2008, 12, 3, 399-410). The geometry of the vicinal syn oxygen functionalized group allows fast and clean cleavage under standard aqueous ammonia deprotection conditions. As shown in FIG. 3, the structure is “pre-organized” with the vicinal hydroxyl groups on the same side of a rigid ring system. When the base sensitive succinate linkage is hydrolyzed, dephosphorylation and release of 3′-hydroxy oligonucleotides occurs.

The methods and systems described herein comprise a solid support system comprising a coated platinum electrode combined with a universal linker molecule that is based on the UNYLINKER™ or UNYSUPPORT™ system, represented herein by formula (II), and is released with aqueous ammonia (Guzaev, 2003, J Am Chem Soc, 125(9):2380-1) or anhydrous methylamine gas (U.S. Pat. No. 7,202,264), the contents of each of these references is herein incorporated by reference in their entireties.

Methods for synthesizing the universal linker therein can be found, for example, in Guzaev, 2003 and Yagodkin, 2011, each of which is herein incorporated by reference in its entirety, specifically with respect to methods of making the universal linker molecules.

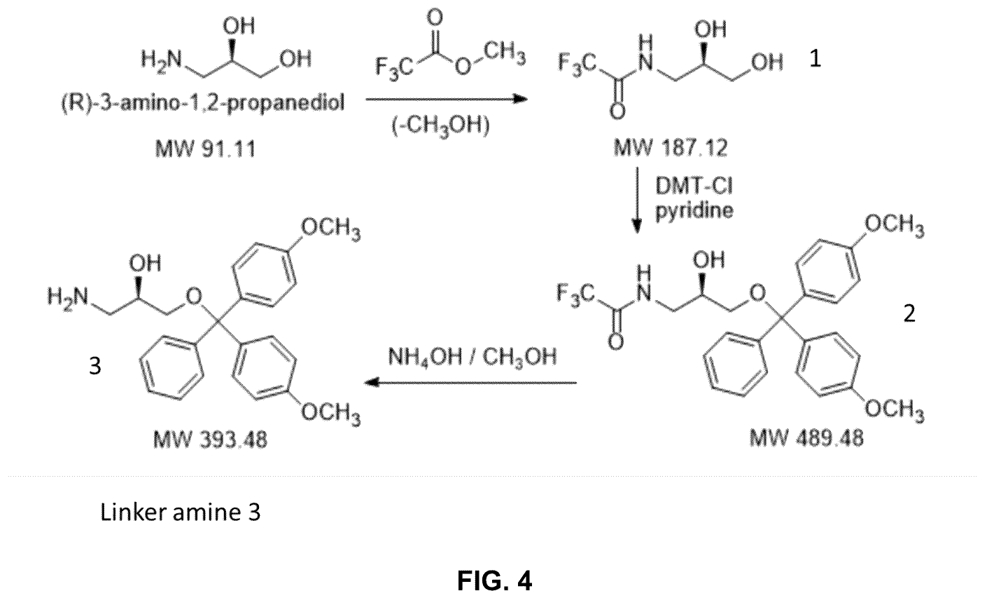

In one embodiment, the linker based on the Universal Support III system starts from the pure (R) or (S)-isomers of 3-amino-1,2-propanediol as shown in FIG. 4. Previous synthesis of the linker system used a racemic mixture of the compound (viscous syrup, bp 264-265 at 739 mm Hg) whereas the pure isomers are solids (mp 54-56 degrees C.). The inventors used the surprisingly inexpensive (R) isomer as a starting material for synthesis of the required linkers. The single stereoisomer at the secondary hydroxyl carbon simplified downstream synthesis of the many intermediates required for synthesis of the universal linker phosphoramidites. For example, the universal linker phosphoramidites described herein are mixtures of 2 diastereomers, whereas previous efforts produced mixtures of 4 diastereomers. Although some physical properties differ, the single isomer universal linkers perform identically to the mixed isomers for release of 3′—OH unmodified oligonucleotides, and we claim both single and mixed isomer structures.

In one embodiment, the methods and systems provided herein comprise a solid support system comprising a coated platinum electrode combined with a universal linker molecule that is based on the Universal Support III system. Methods for synthesizing the universal linker therein can be found, for example, in Azhayev, 2001 and Yagodkin, 2011, each of which is herein incorporated by reference, and specifically with respect to methods of making the universal linker molecule. As noted above, they used mixed stereoisomers but chemical preparations are similar for single isomers.

In one embodiment, the solid support system comprises the universal linker set forth in Formula (I):

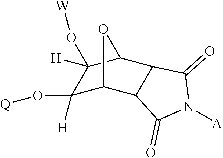

wherein, when A is a linking moiety attached to a coated platinum electrode comprising a substituted or unsubstituted aliphatic group, a substituted or unsubstituted aliphatic ether, a substituted or unsubstituted heteroalkyl group, a substituted or unsubstituted aromatic group, a substituted or unsubstituted heterocyclic group, one of W or Q is a blocking group that is cleavable under basic or neutral conditions, while the other of W or Q is H, or a blocking group that is cleavable under acidic conditions; or
wherein, when A is H, a substituted or unsubstituted aliphatic group, a substituted or unsubstituted aliphatic ether, a substituted or unsubstituted heteroalkyl group, a substituted or unsubstituted aromatic group, a substituted or unsubstituted heterocyclic group, one of Q or W is a linker moiety attached to a coated platinum electrode that is cleavable under basic or neutral conditions, while the other of W or Q is H, or a blocking group that is cleavable under acidic conditions.

In another embodiment, the solid support system comprises the universal linker set forth in Formula (II):

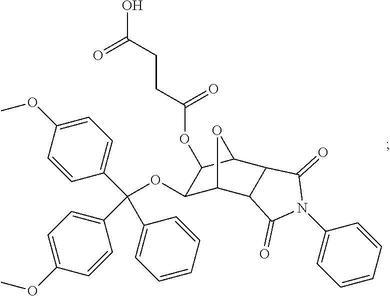 [SMILES: COc1ccc(C(OC2C(OC(=O)CCC(=O)O)C3OC2C2C(=O)N(c4ccccc4)C(=O)C32)(c2ccccc2)c2ccc(OC)cc2)cc1]

In another embodiment, the solid support system comprises the universal linker (mixed and single isomers) set forth in Formula (III):

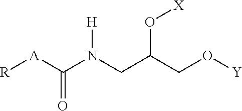

wherein
R is alkyl, aryl, heteroalkyl, or heteroaryl attached to platinum electrode or other base material;
A is NH, O, S, alkyl, or aryl;
X is acyl, aroyl, or silyl; and
Y is dimethoxytrityl group or a protecting group removable under acidic conditions.

In another embodiment, the solid support system comprises the universal linker (mixed and single stereoisomers) set forth in Formula (IV):

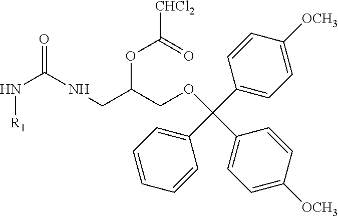

R₁ is alkyl, aryl, heteroalkyl, or heteroaryl attached to platinum electrode or other base material.

In another embodiment, the solid support system comprises the universal linker molecule (mixed and single stereoisomers) set forth in Formula (V):

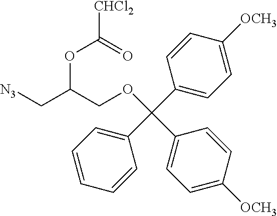

In this embodiment, an amine containing solid support is treated with the azide to form urea bonds (see FIG. 2). This method has been used successfully for synthesis of USIII (Yagodkin 2011). During immobilization, the dichloroacetyl protecting groups likely comes off, and the published protocol re-caps with 1,1′-Carbonyldiimidazole (CDI) activated dichloroacetic acid before use in DNA synthesis.

In other embodiments herein, the universal linker is a phosphoramidite.

In another embodiment, the solid support system comprises the universal linker (mixed and single stereoisomers) set forth in Formula (VI):

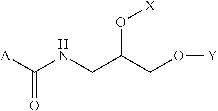

wherein
A is

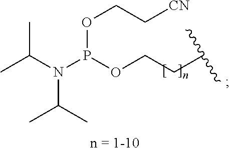

X is acyl, aroyl, or silyl; and
Y is dimethoxytrityl group or a protecting group removable under acidic or neutral conditions.

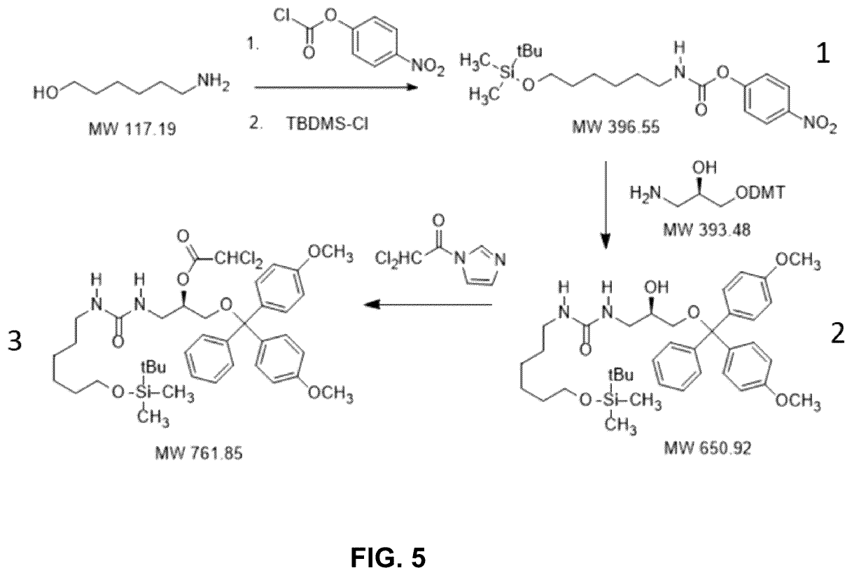

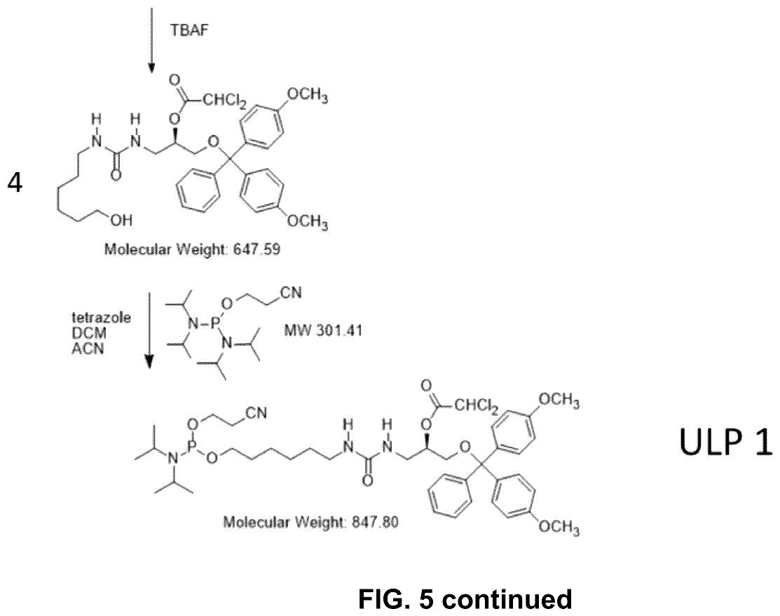

In another embodiment, the solid support system comprises the universal linker (mixed and single stereoisomers) set forth in Formula (VII). Synthesis is described in FIG. 5.

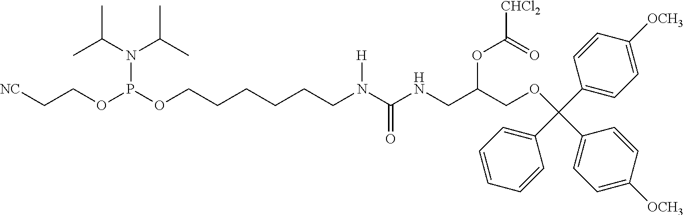

In other embodiments herein, the universal linker is a phosphoramidite.

In one embodiment, the solid support system comprises the universal linker (mixed and single stereoisomers) set forth in Formula (VIII):

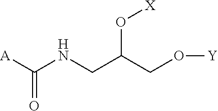

wherein
A is

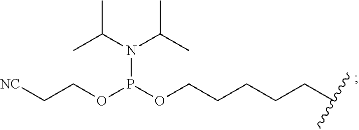 [SMILES: *CCCCCOP(OCCC#N)N(C(C)C)C(C)C]

X is acyl, aroyl, or silyl; and
Y is dimethoxytrityl group or a protecting group removable under acidic or neutral conditions.

In another embodiment, the solid support system comprises the universal linker (mixed and single stereoisomers) set forth in Formula (IX):

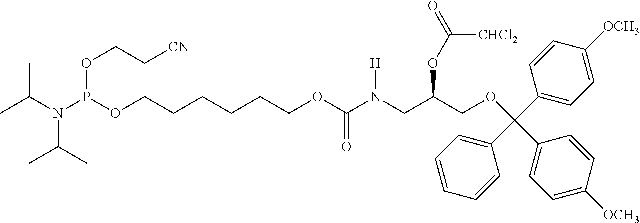

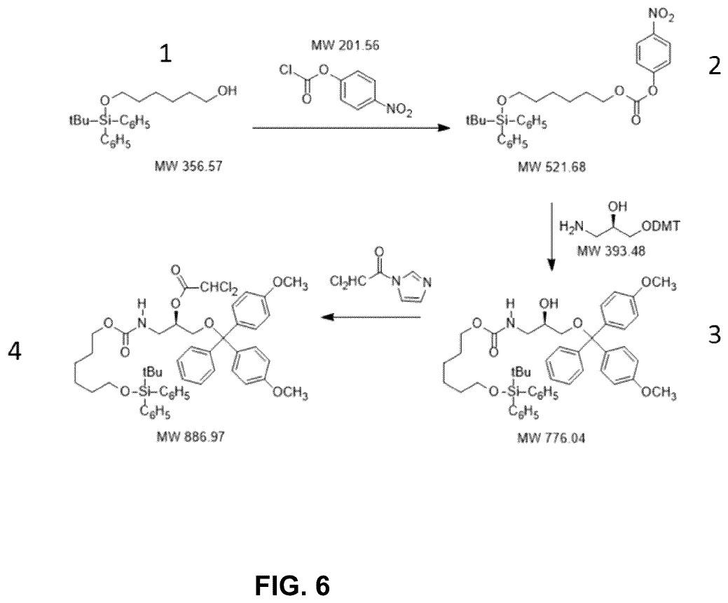

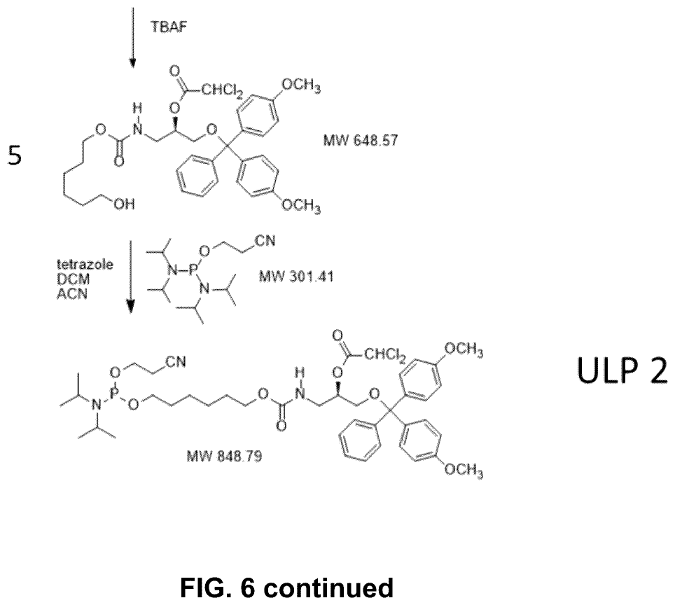

The above linker structure shows a previously unreported carbamate bond between the aliphatic linker and the protected aminopropanediol structure. Synthesis is described in FIG. 6.

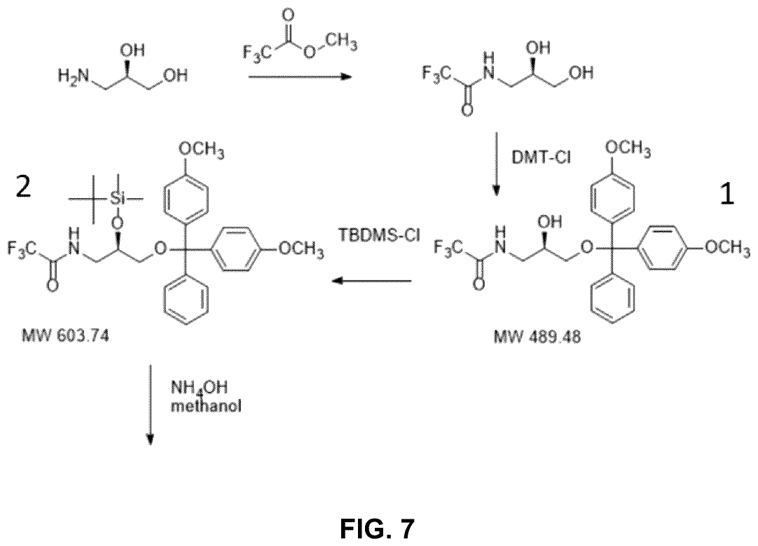

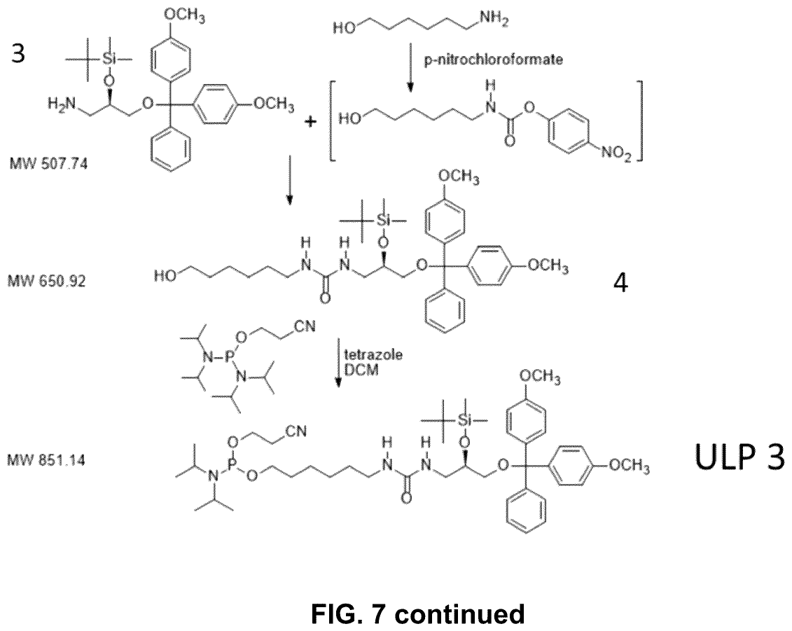

In another embodiment, the solid support system comprises the universal linker (mixed and single stereoisomers) set forth in Formula (X). Synthesis is described in FIG. 7.

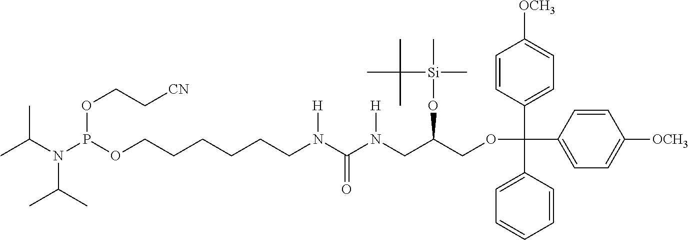 [SMILES: COc1ccc(C(OC[C@@H](CNC(=O)NCCCCCCOP(OCCC#N)N(C(C)C)C(C)C)O[Si](C)(C)C(C)(C)C)(c2ccccc2)c2ccc(OC)cc2)cc1]

The aliphatic groups described herein may have between about 1 and about 10 carbons, about 1 and about 8 carbons, about 2 and about 6 carbons, and may be saturated or unsaturated. Suitable aliphatic groups include but are not limited to methane, acetylene, ethylene, ethane, propyne, propene, propane, 1,2-butadiene, 1-butyne, 1-butene, butane, n-pentyl, nonyl, or combinations thereof.

The lower alkyl groups described herein may have 1 to 6 carbons. For example, a lower alkyl group includes but is not limited to methyl, ethyl, n-propyl, isopropyl, n-butyl, t-butyl, i-butyl, or n-hexyl groups.

The aromatic organic groups described herein may be cyclic carbon chains, alternatively defined according to the Hückel Rule. Aromatic organic groups include but are not limited to benzenes, phenyl groups, aniline, acetophenone, benzaldehyde, benzoic acid, benzonitrile, styrene, ortho-xylene, or combinations thereof.

The lower alcohol groups described herein may be alcohols that are soluble in water, for example, methanol, ethanol, and propanol.

The heteroaromatic groups described herein may be aromatic compounds that contain heteroatoms (e.g., O, N, S) as part of the cyclic conjugated [symbol] system.

The heterocyclic groups described herein, may be substituted or unsubstituted, may be cyclic groups with at least two different types of atoms. Heterocyclic groups generally comprise carbon and nitrogen, sulfur, or oxygen, and may be 3, 4, 5, 6, 7, or 8 member rings. Examples of saturated heterocyclic groups include but are not limited to, aziridine, oxirane, thiirane, azetidine, oxetane, thietane, pyffolidine, oxolane, thiolane, piperdine, oxane, thiane, azepane, oxepane, thiepane, azocane, oxocane, thiocane, azonane, oxonane, and thionane. Examples of unsaturated heterocyclic groups include but are not limited to azirine, oxirene, thiirene, azete, ozete, thiete, pyrrole, furan, thiophene, pyridine, pyran, thipyran, azepine, oxepine, thiepine, azocine, oxocine, thiocine, azonine, oxonine, and thionine.

The nucleosidyl moieties described herein may be a group formed by the loss of —OH from a nucleoside molecule. Nucleoside molecules include but are not limited to cytidine, uridine, adenosine, guanosine, thymidine, and inosine.

The oligonucleotidyl groups may be short strands of DNA or RNA. For example, 1-250 nucleotides (or ribonucleotides) in length.

Provided herein are methods for oligonucleotide synthesis on a solid support medium. The term “oligonucleotide” as used in this document has its conventional meaning. One non-limiting aspect, the term “oligonucleotide” is generic to polydeoxynucleotides (containing 2-deoxy-D-ribose), to polyribonucleotides (containing D-ribose), to any other type of polynucleotide which is an N-glycoside of a purine or pyrimidine base, and to other polymers containing non-nucleotidic backbones, providing that the polymers contain nucleobases in a configuration which allows for base pairing and base stacking, such as is found in DNA and RNA. It will be appreciated that, as used herein, the terms “nucleoside” and “nucleotide” will include those moieties which contain not only the known purine and pyrimidine bases, but also modified purine and pyrimidine bases and other heterocyclic bases which have been modified (these moieties are sometimes referred to collectively as “purine and pyrimidine bases and analogs thereof”). Such modifications include methylated purines or pyrimidines, acylated purines or pyrimidines, and the like. The methods and compositions herein utilize universal linkers to attach the oligonucleotide to the solid support, wherein a non-nucleosidic linker is attached to the solid support material. This approach allows for the same solid support to be used regardless of the sequence of the oligonucleotide to be synthesized.

Novel Universal Linker Phosphoramidite (ULP) reagents are described that can be applied to coated platinum electrodes to allow synthesis of 3′-unmodified nucleic acid strands. Examples of ULPs as embodied herein are set forth in formulas (VII), (VIII). (IX), and (X) and can be prepared using methods described herein. The Universal Linker Phosphoramidites are related to standard nucleotide phosphoramidites disclosed by e.g. Caruthers et al. (U.S. Pat. Nos. 4,415,732; 4,458,066; 4,500,707; 4,668,777; 4,973,679; and 5,132,418), the contents of each are herein incorporated by reference in their entireties.

The sensitivity of the dichloroacetic (DCA) protecting group in the ULP structures can lead to difficulties in synthesis. For example, initial attempts to couple the linker azide (Formula V) to 1-amino-6-hexanol to give a urea bond as described in U.S. Pat. No. 8,779,194 (incorporated herein by reference in its entirety) were unsuccessful because the DCA group did not survive the coupling conditions. As described herein, the present inventors found that silyl groups, such as t-butyldimethylsilyl (TBDMS) could be used to protect the primary alcohol group at an early step, and could be removed at the last reaction step with fluoride reagents, such as aqueous tetrabutylammonium fluoride (TBAF) before conversion to the phosphoramidite. This approach gives high yields and allows synthesis of the target ULPs, such as shown in FIG. 5.

Although 1-amino-6-hexanol was used in the process described in FIG. 5 due mainly to commercial availability, the process is not limited to this linker. Any suitable linker can be used. Other alkyl length and structures may be present in the linker between the reactive phosphoramidite (at the primary hydroxyl group) and the aminopropanediol trigger (DMT protected). For example, aryl or heteroaryl groups may be present in the linker, as long as the groups are unreactive in the DNA synthesis coupling cycle. The choice of linker between amidite group and aminopropanediol trigger is likewise adjustable in other ULPs described in FIGS. 6 and 7

The use of TBDMS was chosen to protect the primary hydroxyl group during the synthesis process shown in FIG. 5 since it can be removed by treatment with fluoride reagents like TBAF. Other silyl protecting groups (e.g., TBDPS) can be used instead of TBDMS. Similarly, other fluoride deprotecting reagents are available and can be used instead of TBAF. In any case, removal of the silyl protecting group must leave the acid sensitive DMT group and the base sensitive DCA group untouched. Other hydroxyl protecting groups like (e.g., benzyl) can be used, and removed under mild hydrogenation conditions. The choice of protecting group on the primary hydroxyl group is likewise adjustable in other ULPs described in FIGS. 6 and 7.

In one embodiment, the desired phosphoramidite is selected from the formula shown in FIG. 5.

In one embodiment, a method is provided to produce a ULP, such as a ULP according to Formula (VII), in which 1-amino-6-hexanol is reacted with p-nitrophenyl chloroformate (4-NPC) and the primary alcohol is further protected with TBDMS. The resulting compound is then coupled to Linker amine 3 (structure shown in FIG. 4) to give TBDMS-protected urea. The S-isomer, or racemic mixture of aminopropanediol Linker amine 3 can also be used, and would likely have comparable rate of cyclization and cleavage as the R-isomer. Only optical rotation of the compounds would be opposite for S-isomer compounds and the racemic compounds would not be optically active. In any case, the presence of urea, amide, or carbamate proton is required to facilitate the rearrangement described in FIG. 2 after hydrolysis of the DCA trigger. The DCA ester can be formed, for example, by activating with carbonyldiimidazole as described earlier (Yagodkin 2011). Other methods for introducing the DCA ester are possible, but we found the CDI method was convenient and give good yields on reasonable scale. Finally, the TBDMS group is removed with TBAF and the alcohol is phosphitylated to give the desired phosphoramidite (e.g., ULP 1). An exemplary process for the production of ULP 1 according to this embodiment is shown in FIG. 5.

In another embodiment, a method is provided to produce a ULP, such as a ULP according to Formula (IX), in which TBDPS-protected 1,6-hexanediol is first prepared and activated with p-nitrophenyl chloroformate (4-NPC). The resulting compound is then coupled with Linker amine 3 to give the TBDPS-protected carbamate. The S-isomer, or racemic mixture of aminopropanediol Linker amine 3 can also be used, and would likely have comparable rate of cyclization and cleavage as the R-isomer. The DCA ester can be formed, for example, by activating with carbonyldiimidazole although other methods are possible. Finally, the TBDPS group is removed with TBAF and the alcohol is phosphitylated to give the desired phosphoramidite (e.g., ULP 2). An exemplary process for the production of ULP 2 according to this embodiment is shown in FIG. 6.

In another embodiment, a method is provided to produce a ULP, such as a ULP according to Formula (X), in which the ULP (e.g., ULP3) is prepared using a fluoride triggered silyl protecting group instead of the base triggered DCA protecting group. According to this embodiment, synthesis starts from the (R) aminopropanediol as shown in FIG. 4, but TBDMS-Cl is used to protect the secondary hydroxyl group (66% yield for 3 steps). The trifluoroacetamide protecting group is then removed with ammonium hydroxide. Reaction of the amine with p-nitrophenyl chloroformate (4-NPC) activated 6-aminohexanol gives a urea bond. The primary alcohol is phosphitylated directly to give the desired phosphoramidite (e.g., ULP 3). An exemplary process for the production of ULP 3 according to this embodiment is shown in FIG. 7.

Any support material suitable for use in oligonucleotide synthesis can be used with the invention. For example, solid supports can be beads, particles, sheets, dipsticks, rods, membranes, filters, fibers (e.g., optical or glass), semiconductor devices, or in any other suitable form. Further suitable solid supports comprise materials including but not limited to borosilicate glass, agarose, sepharose, magnetic beads, polystyrene, polyacrylamide, membranes, silica, semiconductor materials, silicon, organic polymers, ceramic, glass, metal, plastic polycarbonate, polycarbonate, polyethylene, polyethyleneglycol terephthalate, polymethylmethacrylate, polypropylene, polyvinylacetate, polyvinylchloride, polyvinylpyrrolidinone, and soda-lime glass. The substrate body may be in the form of a bead, box, column, cylinder, disc, dish (e.g., glass dish, PETRI dish), fiber, film, filter, microtiter plate (e.g., 96-well microtiter plate), multi-bladed stick, net, pellet, plate, ring, rod, roll, sheet, slide, stick, tray, tube, or vial. The substrate can be a singular discrete body (e.g., a single tube, a single bead), any number of a plurality of substrate bodies (e.g., a rack of 10 tubes, several beads), or combinations thereof (e.g., a tray comprises a plurality of microtiter plates, a column filled with beads, a microtiter plate filed with beads).

The material composition of the solid support materials may be any suitable material, such as polymeric or silica-based support materials. Specific examples include plastic, nylon, glass, silica, metal, metal alloy, polyacrylamide, polyacrylate, polystyrene, cross-linked dextran, and combinations thereof.

### Solid Supports

In an aspect, the support material for oligonucleotide synthesis may comprise a flat (planar) electrode. A flat electrode generates either a divergent or homogeneous field depending on the orientation of the grooved electrodes. The flat electrode can be oriented with the grooved sides of the electrode facing one another to generate a divergent field for use in electro cell fusion. Alternatively, it can be oriented with the flat sides facing each other providing a homogeneous field for electroporation.

The flat electrode may be a dense electrode array comprising a plurality of cells and a surface, where each cell of the plurality of cells includes an anode and a circumferential cathode, where each of the anodes are separately addressable electrodes, and where a porous reaction layer is adsorbed to the surface.

The electrode array devices can be fabricated using standard CMOS technology. This device utilizes alternating array of circular active electrodes and continuous circumferential counter electrodes. In a CMOS process, the semiconductor silicon wafer is fabricated using aluminum wiring and electrodes and then “post-processed” by sputtering another metal. In certain embodiments, the metal is platinum.

Another format is to have a standard electrode array device made with circular electrodes arranged in rows and columns, there are lines separating each “cell” of the electrode array. A cell comprises an electrode and the associated circuitry needed to independently electronically access each electrode individually. In certain embodiments, the wires separating each cell can be raised to the surface of the electrode array (where the electrodes have surface exposure) and function as an array-wide grid of counter electrode for which electrodes are turned on in each electrochemical synthesis step.

The oligonucleotide synthesis may be performed on a support medium comprising a plurality of separately addressable platinum electrodes. The electrodes can be coated using aryldiazonium salts. Aryl diazonium salts are represented by the generic formula R-Ar-N₂⁺X⁻, where R can be any organic group, such as an alkyl or an aryl, and X is an inorganic or organic anion, such as a halogen or tetrafluoroborate. The term “halogen” represents chlorine, fluorine, bromine, or iodine. In one embodiment, a carboxylic acid coating can be applied to the electrode surface using the diazonium salt of aminophenyl acetic acid (APA) and electrochemical reduction (also known as electrodeposition or electrografting). Similar chemistry has been described for coating gold electrodes with phenylethanol groups for DNA synthesis (Levrie, 2018, Jpn. J. Appl. Physics, 04FM01, which is herein incorporated by reference in its entirety).

### Oligonucleotide Synthesis

The standard synthetic methods for oligonucleotides are known in the art (e.g., U.S. Pat. Nos. 5,750,666, 6, 111,086, 6,008,400, and 5,889,136), each of which is incorporated herein by reference in their entirety.

Support bound oligonucleotide synthesis relies on sequential addition of nucleotides to one end of a growing chain. In the present invention, the universal linkers described herein are reacted onto a solid surface support, e.g., platinum coated with an amine for the oligonucleotide synthesis. Typically, a first nucleoside (having protecting groups on any exocyclic amine functionalities present) is attached to the solid support medium and activated phosphite compounds (which also bear appropriate protecting groups) are added stepwise to elongate the growing oligonucleotide. Additional methods for solid-phase synthesis may be found in Caruthers U.S. Pat. Nos. 4,415,732; 4,458,066; 4,500,707; 4,668,777; 4,973,679; and 5,132,418; and Koster U.S. Pat. No. 4,725,677, the contents of each which are incorporated by reference in their entireties.

Electrochemical reagents capable of electrochemically removing protecting groups from chemical functional groups on the molecule are generated at selected electrodes by applying a sufficient electrical potential to the selected electrodes. Removal of a protecting group, or “deprotection,” in accordance with the invention, occurs at selected molecules when a chemical reagent generated by the electrode acts to deprotect or remove, for example, an acid or base labile protecting group from the selected molecules. Silyl protecting groups can be deprotected with a source of fluoride ion. Thus, in some embodiments the chemical reagent is a fluoride reagent. Examples of suitable fluoride reagents include, but are not limited to, tetrabutylammonium fluoride (TBAF), pyridine (HF)x, triethylamine trihydrofluoride (TREAT HF), hydrofluoric acid, tris(dimethylamino)sulfonium difluorotrimethylsilicate (TASF), and ammonium fluoride.

In one embodiment of the present invention, a terminal end of a monomer nucleotide, or linker molecule (i.e., a molecule which “links,” for example, a monomer or nucleotide to a substrate) is provided in accordance with the present invention, which is protected with a protecting group removable by an electrochemically generated reagent. The protecting group(s) is exposed to reagents electrochemically generated at the electrode and removed from the monomer, nucleotide or linker molecule in a first selected region to expose a reactive functional group. The substrate is then contacted with a first monomer or pre-formed molecule, which bonds with the exposed functional group(s). This first monomer or pre-formed molecule may also bear at least one protected chemical functional group removable by an electrochemically generated reagent.

The term “protecting group” (or “blocking group”) as used herein, refers to a labile chemical moiety which is known in the art to protect a hydroxyl, amino or thiol group against undesired reactions during synthetic procedures. Protecting groups as known in the art are described generally in T. H. Greene and P. G. M. Wuts, 1999, Protective Groups in Organic Synthesis, 3rd edition, John Wiley & Sons, New York. Examples of hydroxyl protecting groups include, but are not limited to, benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl (BOC), isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl (Alloc), acetyl (Ac), formyl, chloroacetyl, trifluoroacetyl, methoxyacetyl, phenoxyacetyl, benzoyl (Bz), methyl, t-butyl, 2,2,2-trichloroethyl, 2-trimethylsilyl ethyl, 1,1-dimethyl-2-propenyl, 3-methyl-3-butenyl, allyl, benzyl (Bn), para-methoxybenzyldiphenylmethyl, triphenylmethyl (trityl), 4,4′-dimethoxytriphenylmethyl (DMT), substituted or unsubstituted 9-(9-phenyl)xanthenyl (pixyl), tetrahydrofuryl, methoxymethyl, methylthiomethyl, benzyloxymethyl, 2,2,2-trichloroethoxymethyl, 2-(trimethylsilyl)ethoxymethyl, methanesulfonyl, para-toluenesulfonyl, trimethylsilyl, triethylsilyl, and triisopropylsilyl. In some embodiments, the protecting group is DMT.

In some embodiments, the hydroxyl protecting group is a silyl protecting group. Examples of silyl protecting groups include, but are not limited to, 2-(trimethylsilyl)ethoxycarbonyl, 2-trimethylsilyl ethyl, 2-(trimethylsilyl)ethoxymethyl, trimethylsilyl (TMS), triethylsilyl (TES), triisopropylsilyl (TIPS), isopropyldimethylsilyl (IPDMS), diethylisopropylsilyl (DEIPS), t-butyldimethylsilyl (TBS), t-butyldiphenylsilyl (TBDPS), tetraisopropyldisiloxanylidene (TIPDS), di-t-butylsilylene (DTBS), and t-butyldimethylsilyl (TBDMS).

The monomers or pre-formed molecules can then be deprotected in the same manner to yield a second set of reactive chemical functional groups. A second monomer or pre-formed molecule, which may also bear at least one protecting group removable by an electrochemically generated reagent, is subsequently brought into contact with the substrate to bond with the second set of exposed functional groups. Any unreacted functional groups can optionally be capped at any point during the synthesis process. The deprotection and bonding steps can be repeated sequentially at this site on the substrate until polymers or oligonucleotides of a desired sequence and length are obtained.

The substrate having one or more molecules bearing at least one protected chemical functional group bonded thereto may be proximate an array of electrodes, which array is in contact with a buffering or scavenging solution. Following application of an electric potential to selected electrodes in the array sufficient to generate electrochemical reagents capable of deprotecting the protected chemical functional groups, molecules proximate the selected electrodes are deprotected to expose reactive functional groups, thereby preparing them for bonding. A monomer solution or a solution of pre-formed molecules, such as proteins, nucleic acids, polysaccharides, and porphyrins, is then contacted with the substrate surface and the monomers or pre-formed molecules bond with the deprotected chemical functional groups.

The methods described herein may further comprise reacting said monomer-functionalized support medium with a capping agent; and optionally treating said monomer-functionalized support medium with an oxidizing agent.

Following oligonucleotide synthesis, the oligonucleotides can be released from or immobilized onto the solid support medium. These methods may comprise a step of treating the oligonucleotide with a reagent effective to cleave the oligonucleotide from the support medium, preferably from the linker attached to the support medium. In some such embodiments, the treating of the oligonucleotide with a reagent effective to cleave the oligonucleotide removes protecting groups present on the oligonucleotide. In some embodiments, the cleaved oligonucleotide has a 3′ unmodified terminal hydroxyl group at the site of cleavage. In various embodiments, solid support medium is treated with anhydrous ammonia for a period of time sufficient to cleave the oligonucleotide.

The cleaved oligonucleotide may then be prepared by procedures known in the art, for example by size exclusion chromatography, high performance liquid chromatography (e.g., reverse-phase HPLC), differential precipitation, etc. In some embodiments according to the present invention, the oligonucleotide is cleaved from a solid support medium while the 5′—OH protecting group is still on the ultimate nucleoside. This so-called DMT-on (or trityl-on) oligonucleotide is then subjected to chromatography, after which the DMT group is removed by treatment in an organic acid, after which the oligonucleotide is de-salted and further purified to form a final product.

In some embodiments, immobilized oligonucleotides can be prepared from the Universal support linker system described herein. The oligonucleotide-bound support structure is first treated with a 20% solution of t-butylamine in acetonitrile for 1 hour (Chang and Horn, 1999, Nucleosides and Nucleotides, 2006, pp 1205-6) to remove cyanoethyl groups and the acrylonitrile side products. The resulting phosphodiester is stable and does not cleave when the dichloroacetate group is hydrolyzed with aqueous ammonia or AMA (1:1, 37% ammonium hydroxide:40% methylamine) treatment (see, e.g., FIG. 3). After cleavage of protecting groups is complete, the chip is washed and oligonucleotides remain immobilized over the electrodes.

### Use of Oligonucleotides

The methods and compositions provided herein are useful for genome editing libraries such as CRISPR gRNA screening libraries and shRNA screening libraries, targeted sequencing such as hybrid-capture or molecular inversion probes (MIPs), mutagenesis libraries, generation of oligos for in situ hybridization applications, and generation of pools of oligos for DNA data storage.

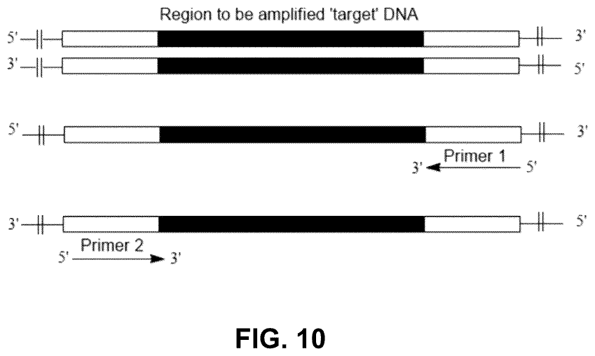

A common application for automated DNA synthesis is for production of PCR primers. PCR uses a pair of custom primers to direct DNA elongation toward each-other at opposite ends of the sequence being amplified. These primers are typically between 18 and 24 bases in length and should code for only the specific upstream and downstream sites of the sequence being amplified as shown in FIG. 10.

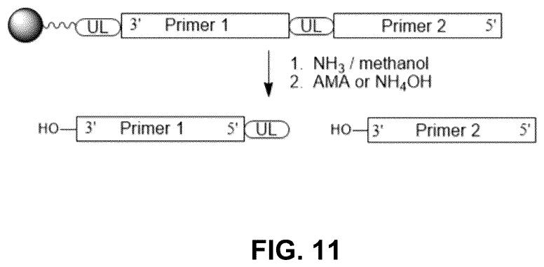

The Universal Linker Phosphoramidites described herein may be used to make 2 primers in a single synthesis column as shown in FIG. 11. The ULP introduces a spacer between the Primer 1 and Primer 2 DNA sequences as shown in FIG. 11. After “trityl-off” DNA synthesis, the solid support can be treated with 4M ammonia/MeOH. Primer 1 sequence is released from the solid support as usual with 3′-hydroxy group. The universal linker spacer is simultaneously cleaved to give Primer 2 sequence with a 3′-hydroxy group. Removal of protecting groups with aqueous ammonia or AMA generates a 1:1 mixture of Primer 1 and Primer 2. Evaporation leaves a 1:1 mix of primers, along with contaminating failure sequences and removed protecting groups.

Usually PCR primers are purified by removing the 5′-trityl group on the oligo and simply “desalting” using a gel filtration column. Gel filtration is the separation of the components of a mixture on the basis of molecular size and is one of the simplest forms of chromatography for oligonucleotide purification. Cleaved protecting groups and short truncated sequences are retained in the gel matrix while larger oligonucleotide molecules elute quickly through the gel filtration column. Since Primer 1 and Primer 2 are about the same length, they elute in the same fraction. Concentration of the 1:1 mix of primers is determined by 260 nm absorbance using the combined extinction coefficients of the 2 oligos. The 1:1 mixture can be used directly in PCR without having to separately dissolve the 2 oligos, determine the concentration of each primer, calculate the volume of each to achieve a 1:1 mixture, pipetting and mixing the required volumes, and re-drying the mixture. Therefore much labor is saved by automatically synthesizing a 1:1 mixture of both primers and purifying in a single operation.

Using phosphoramidite DNA synthesis chemistry molecules can be synthesized on the surface of a solid support substrate in a step-by-step reaction proceeding, generally, in the 3′ to 5′ direction and consisting of (1) a detritylation step to remove a protecting group from the previously added nucleoside. (2) a coupling of the next nucleoside to the growing DNA oligomer, (3) oxidation to convert the phosphite triester intermediate into a more stable phosphate triester. (4) irreversibly capping any unreacted 5′ hydroxyls groups. Without being bound by theory, capping unreacted 5′ hydroxyl groups can help prevent synthesized sequences having a deletion relative to preselected nucleic acid sequences by avoiding continued polymerization from such 3′ hydroxyl groups in subsequent cycles. The cycle can be repeated to add the next base. Solid supports may comprise a variety of units, such as beads, including without limitation highly porous polymeric beads, glass or silica beads including, but not limited to fused silica (amorphous pure silica), quartz (crystalline pure silica), metals (titanium, e.g., titanium nitride, or platinum), or other any other suitable beads described herein or otherwise known in the art, which can be packed into a chamber or column, to which the synthesis reagents are delivered. The methods, devices and compositions described herein can be used to scale nucleic acid synthesis methods using microfluidic approaches.

Trityl-off oligonucleotide synthesis refers to the use of a 5′-O′trityl group that protects the 5′-hydroxyl group of the target oligonucleotides during the coupling and oxidation steps. Upon synthesis completion, the trityl group can be cleaved from the target oligonucleotides (e.g., “trityl off sequences) with acid.

The acidic conditions may include pH at about 1 to about 6.9, about 2 to about 6.9, about 3 to about 6.9, about 4 to about 6.9, about 5 to about 6.9, and about 6 to about 6.9.

The neutral conditions may include pH at about 6.9 to about 7, about 7, about 7 to about 7.1, about 7 to about 7.2, about 7 to about 7.3, about 7 to about 7.4, and about 7 to about 7.5.

### Definitions

“Phosphoramidite” (RO)₂PNR₂ refers to a monoamide of a phosphite diester. Features of phosphoramidites may include their high reactivity towards nucleophiles catalyzed by weak acids, e.g., triethylammonium chloride or 1H-tetrazole. In these reactions, the incoming nucleophile may replace the NR₂ moiety.

“Aliphatic” refers to open-chain hydrocarbons radical, whether straight or branched, which contains no rings of any type, and cyclic hydrocarbons radical if they are not aromatic.

“Aliphatic either” refers to an ether in the molecule of which there are no aryl groups on the ether group.

“Aromatic” refers to mono- and polycyclic aromatic hydrocarbons radical.

“Acyl” refers to a moiety derived by the removal of one or more hydroxyl groups from an oxoacid including inorganic acids. It may contain a double-bonded oxygen atom and an alkyl group (R—C═O).

“Aroyl” refers to any univalent radical R—CO— derived from an aromatic carboxylic acid.

“Vicinal diol” refers to two hydroxyl groups occupying vicinal positions, i.e., they are attached to adjacent atoms.

“Alkyl” refers to a straight or branched hydrocarbon chain radical consisting solely of carbon and hydrogen atoms, containing no unsaturation, having from one to twelve carbon atoms, preferably one to eight carbon atoms or one to six carbon atoms, and which is attached to the rest of the molecule by a single bond, e.g., methyl, ethyl, n-propyl, 1-methylethyl(iso-propyl), n-butyl, n-pentyl, 1,1-dimethylethyl(t-butyl), and the like.

“Heteroalkyl” refers to an alkyl group substituted by one or more of the following groups: alkyl, alkenyl, halo, haloalkyl, cyano, aryl, cycloalkyl, heterocyclyl, heteroaryl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)(S(O)ₜR¹⁶) (where t is 1 to 2), —S(O)ₜOR¹⁶, —SR¹⁶ (where t is 1 to 2), —S(O)ₜR¹⁶ (where t is 0 to 2), and —S(O)ₜN(R¹⁴)₂ (where t is 1 to 2), where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, cycloalkyl, cycloalkylalkyl, aryl, aralkyl, heterocyclyl, heterocyclylalkyl, heteroaryl or heteroarylalkyl.

“Aryl” refers to aromatic monocyclic or multicyclic hydrocarbon ring system consisting only of hydrogen and carbon and containing from six to nineteen carbon atoms, preferably six to ten carbon atoms, where the ring system may be partially saturated. Aryl groups include, but are not limited to groups such as fluorenyl, phenyl and naphthyl. Unless stated otherwise specifically in the specification, the term “aryl” or the prefix “ar—” (such as in “aralky!”) is meant to include aryl radicals optionally substituted by one or more substituents selected from the group consisting of alkyl, alkenyl, alkynyl, halo, haloalkyl, cyano, nitro, aryl, aralkyl, cycloalkyl, cycloalkylalkyl, heterocyclyl, heterocyclylalkyl, heteroaryl, heteroarylalkyl, —R¹⁵-OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶) (where t is 1 to 2). —R¹⁵—S(O)ₜOR¹⁶ (where t is 1 to 2), —R¹⁵—SR¹⁶, —R¹⁵—S(O)ₜR¹⁶ (where t is 0 to 2), and -—R¹⁵—S(O)ₜN(R¹⁴)₂ (where t is 1 to 2), where each R¹⁴ is independently hydrogen, alkyl, haloalkyl, cycloalkyl, cycloalkylalkyl, aryl, aralkyl, heterocyclyl, heterocyclylalkyl, heteroaryl, or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl, or heteroarylalkyl.

“Heterocyclyl” refers to a stable 3- to 18-membered non-aromatic ring radical which consists of carbon atoms and from one to five heteroatoms selected from the group consisting of nitrogen, oxygen and sulfur. For purposes of this invention, the heterocyclyl radical may be a monocyclic, bicyclic or tricyclic ring system, which may include fused or bridged ring systems, which may be partially unsaturated; and the nitrogen, carbon or sulfur atoms in the heterocyclyl radical may be optionally oxidized; the nitrogen atom may be optionally alkylated/substituted; and the heterocyclyl radical may be partially or fully saturated. Examples of such heterocyclyl radicals include, but are not limited to, dioxolanyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl, homopiperidinyl, homopiperazinyl, and quinuclidinyl. Unless stated otherwise specifically in the specification, the term “heterocyclyl” is meant to include heterocyclyl radicals as defined above which are optionally substituted by one or more substituents selected from the group consisting of alkyl, alkenyl, halo, haloalkyl, cyano, oxo, thioxo, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂—R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶) (where t is 1 to 2), —R¹⁵—S(O)ₜOR¹⁶ (where t is 1 to 2), —R¹⁵—SR¹⁶, —R¹⁵—S(O)ₜR¹⁶ (where t is 0 to 2), and —R¹⁵—S(O)ₜN(R¹⁴)₂ (where t is 1 to 2), where each R¹⁴ is independently hydrogen, alkyl, haloalkyl, cycloalkyl, cycloalkylalkyl, aryl, aralkyl, heterocyclyl, heterocyclylalkyl, heteroaryl, or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl, or heteroarylalkyl, and where each of the above substituents is unsubstituted.

“Heteroaryl” refers to a 5- to 18-membered aromatic ring radical which consists of carbon atoms and from one to five heteroatoms selected from the group consisting of nitrogen, oxygen and sulfur. For purposes of this invention, the heteroaryl radical may be a monocyclic, bicyclic or tricyclic ring system, which may include fused or bridged ring systems, which may be partially saturated; and the nitrogen, carbon or sulfur atoms in the heteroaryl radical may be optionally oxidized; the nitrogen atom may be optionally alkylated/substituted. Examples include, but are not limited to, azepinyl, acridinyl, benzimidazolyl, benzthiazolyl, benzindolyl, benzothiadiazolyl, benzonaphthofuranyl, benzoxazolyl, benzodioxolyl, benzodioxinyl, benzopyranyl, benzopyranonyl, benzofuranyl, benzofuranonyl, benzothienyl, benzothiophenyl, benzotriazolyl, benzo[4,6]imidazo[1,2-a]pyridinyl, carbazolyl, cinnolinyl, dibenzofuranyl, furanyl, furanonyl, isothiazolyl, imidazolyl, indolyl, indazolyl, isoindolyl, indolinyl, isoindolinyl, indolizinyl, isoxazolyl, naphthyridinyl, oxadiazolyl, 2-oxoazepinyl, oxazolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, thiazolyl, thiadiazolyl, triazolyl, tetrazolyl, triazinyl, and thiophenyl. Unless stated otherwise specifically in the specification, the term “heteroaryl” is meant to include heteroaryl radicals as defined above which are optionally substituted by one or more substituents selected from the group consisting of alkyl, alkenyl, alkynyl, halo, haloalkyl, cyano, oxo, thioxo, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶) (where t is 1 to 2), —R¹⁵—S(O)ₜOR¹⁶ (where t is 1 to 2), —R¹⁵—SR¹⁶, —R¹⁵—S(O)ₜR¹⁶ (where t is 0 to 2), and —R¹⁵—S(O)ₜN(R¹⁴)₂ (where t is 1 to 2), where each R¹⁴ is independently hydrogen, alkyl, haloalkyl, cycloalkyl, cycloalkylalkyl, aryl, aralkyl, heterocyclyl, heterocyclylalkyl, heteroaryl, or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl, or heteroarylalkyl.

“Silyl either” refers to a group of chemical compounds, which contain a silicon atom covalently bonded to an alkoxy group. The general structure is R₁R₂R₃Si—O—R₄, where R₄ is an alkyl group or an aryl group. Since R₁R₂R₃ can be combinations of differing groups, which can be varied in order to provide a number of silyl ethers, this group of chemical compounds provides a wide spectrum of selectivity for protecting group chemistry. Silyl ethers may include, but not limited to, trimethylsilyl (TMS), Triethylsilyl (TES), tert-butyldiphenylsilyl (TBDPS), tert-butyldimethylsilyl (TBS/TBDMS), and triisopropylsilyl (TIPS).

### EXAMPLES

### Example 1: Versatile Universal Linker Cleavage After DNA Synthesis

After DNA synthesis, the Universal linker structure can either be cleaved from the electrode surface with 4-9M ammonia in anhydrous methanol for ULP1 and ULP2, or with 1.6% (v/v) TEA:3HF in anhydrous methanol for ULP3. Alternatively, the Universal linker can be immobilized to the surface using 20% t-butylamine or 10% 1,8-Diazabicyclo(5.4.0)undec-7-ene (DBU) in acetonitrile (ACN) as shown for ULP1 and ULP3 in FIG. 2.

Treatment of ULP1 and ULP 2 with anhydrous ammonia in methanol rapidly cleaves the dichloroacetyl group and dephosphorylation releases oligo. Treatment of ULP3 with TEA:3HF cleaves the TMDMS protecting group which also results in dephosphorylation and oligo release. Treatment of ULP1, ULP2 and ULP3 with t-butylamine or DBU in ACN rapidly cleaves the cyanoethyl group and immobilizes the oligonucleotide to the surface.

After the oligonucleotides are released from support, the solution is removed from the solid support and combined in a screw cap tube with aqueous ammonia (37%) or AMA (1:1, 37% ammonium hydroxide:40% methylamine). After heating, the fully deprotected oligonucleotides are dried in vacuo and purified using standard methodologies.

For immobilized oligonucleotides, the solid support is first treated with a 20% solution of t-butylamine in acetonitrile for 1 hour (see, for example, Chang and Horn, 1999, Nucleosides and Nucleotides, 2006, pp 1205-6) to remove cyanoethyl groups and the acrylonitrile side products. The resulting phosphodiester is stable and does not cleave when the dichloroacetate group is hydrolyzed with aqueous ammonia or AMA treatment. After cleavage of protecting groups is complete, the solid support is washed and oligos remain immobilized over the electrodes.

### Example 2: Linker Amine 3 (R) Isomer

Linker amine 3 (FIG. 4) is isolated as a single stereoisomer using a modification of the published procedure for the racemic mixture (see, for example, Azhayev, 2001). Trifluoroacetamide protection of (R)-3-amino-1,2-propanediol with methyl trifluoroacetate was followed by reaction with a limiting amount of dimethoxytrityl chloride in pyridine. The product was easily isolated by extractive workup as the excess trifluoroacetate was easily washed away with water. Trace impurities were removed after ammonium hydroxide deprotection by precipitation from hexanes to give (R) Linker amine 3 in 91% yield as a white solid. The racemic mixture was previously reported as a colorless syrup. The intermediate compounds produced in the process of preparing Linker Amine 3 (FIG. 4) are shown below:
1. (R)—N—(2,3-dihydroxypropyl)-2,2,2-trifluoroacetamide. (MW 187.12) ¹H NMR (CD₃CN)
2. (R)—N—(3-(bis(4-methoxyphenyl)(phenyl)methoxy)-2-hydroxypropyl)-2,2,2-trifluoroacetamide (MW 489.48)
3. (R)—1-amino-3-(bis(4-methoxyphenyl)(phenyl)methoxy)propan-2-ol (MW 393.48)

### Example 3: Universal Linker Phosphoramidite (Urea Bond)—ULP 1

The sensitivity of the dichloroacetic (DCA) protecting group in the ULP structures leads to difficulties in synthesis. We found that t-butyldimethylsilyl (TBDMS) could be used to protect the primary alcohol group at an early step, and could be removed at the last reaction step with aqueous tetrabutylammonium fluoride (TBAF) before conversion to the phosphoramidite. This approach gave high yields and allowed synthesis of the target ULP shown in FIG. 5. We first tried the more stable t-butyldiphenylsilyl (TBDPS) as shown in FIG. 6. TBDPS worked, but longer reaction time in aqueous tetrabutylammonium fluoride (TBAF) gave some hydrolysis of the DCA.

We first attempted to couple the linker azide (structure V) to 1-amino-6-hexanol to give a urea bond as described in U.S. Pat. No. 8,779,194. However, we found that the dichloroacetyl group did not survive the coupling conditions. Instead, we reacted 1-amino-6-hexanol with p-nitrophenyl chloroformate (4-NPC) and further protected the primary alcohol with TBDMS. This compound was coupled to Linker amine 3 (structure shown in FIG. 4) to give the TBDMS protected urea. The DCA ester was formed by activating with carbonyldiimidazole as described earlier (Yagodkin 2011). Finally, the TBDMS group is removed with TBAF and the alcohol is phosphitylated to give the desired phosphoramidite (ULP 1). The intermediate compounds produced in the process of preparing ULP1 (FIG. 5) are shown below:
1. 4-nitrophenyl 6-(tert-butyldimethylsilyloxy)hexylcarbamate
2. (R)-1-(3-(bis(4-methoxyphenyl)(phenyl)methoxy)-2-hydroxypropyl)-3-(6-(tert-butyldimethylsilyloxy)hexyl)urea
3. (R)-1,1-bis(4-methoxyphenyl)-16,16,17,17-tetramethyl-7-oxo-1-phenyl-2,15-dioxa-6,8-diaza-16-silaoctadecan-4-yl 2,2-dichloroacetate
4. (R)-1-(bis(4-methoxyphenyl)(phenyl)methoxy)-3-(3-(6-hydroxyhexyl)ureido) propan-2-yl 2,2-dichloroacetate

### Example 4: Universal Linker Phosphoramidite (Carbamate Bond)—ULP2

ULP2 is prepared with a carbonate structure using a similar scheme to that described in Example 3 (see FIG. 6). In this example, TBDPS protected 1,6-hexanediol is first prepared and activated with p-nitrophenyl chloroformate (4-NPC). This compound is coupled with Linker amine 3 to give the TBDMS-protected carbamate. The DCA ester is formed by activating with carbonyldiimidazole as usual. Finally, the TBDPS group is removed with TBAF and the alcohol is phosphitylated to give the desired phosphoramidite (ULP2). The intermediate compounds produced in the process of preparing ULP2 (FIG. 6) are shown below:
1. 6-(tert-butyldiphenylsilyloxy)hexan-1-ol
2. 6-(tert-butyldiphenylsilyloxy)hexyl 4-nitrophenyl carbonate
3. (R)-6-(tert-butyldiphenylsilyloxy)hexyl 3-(bis(4-methoxyphenyl)(phenyl)methoxy)-2-hydroxypropylcarbamate
4. (R)-1,1-bis(4-methoxyphenyl)-17,17-dimethyl-7-oxo-1,16,16-triphenyl-2,8, 15-trioxa-6-aza-16-silaoctadecan-4-yl 2,2-dichloroacetate
5. (R)-1-(bis(4-methoxyphenyl)(phenyl)methoxy)-3-((6-hydroxyhexyloxy)carbonylamino)propan-2-yl 2,2-dichloroacetate

### Example 5: Universal Linker Phosphoramidite (TBDMS Protected)—ULP3

ULP3 is prepared using a fluoride triggered silyl protecting group instead of the base triggered DCA protecting group (FIG. 7). The synthesis starts from the (R) aminopropanediol as shown in FIG. 4, but TBDMS-Cl is used to protect the secondary hydroxyl group (66% yield for 3 steps). The trifluoroacetamide protecting group is removed with ammonium hydroxide. Reaction of the amine with p-nitrophenyl chloroformate (4-NPC) activated 6-aminohexanol gives a urea bond. In this example, the primary alcohol is phosphitylated directly to give the desired phosphoramidite (ULP 3). The intermediate compounds produced in the process of preparing ULP3 (FIG. 7) are described below:
1. (R)—N—(3-(bis(4-methoxyphenyl)(phenyl)methoxy)-2-hydroxypropyl)-2,2,2-trifluoroacetamide (MW 489.48)
2. (R)—N—(3-(bis(4-methoxyphenyl)(phenyl)methoxy)-2-(tert-butyldimethylsilyloxy)propyl)-2,2,2-trifluoroacetamide (MW 603.74) TLC (ethyl acetate-hexane [1:5]) R_{f}=0.59; mass spectrum (El mode) m/z 604 [M+H]⁺.
3. (R)-3-(bis(4-methoxyphenyl)(phenyl)methoxy)-2-(tert-butyldimethylsilyloxy)propan-1-amine (MW 507.74) TLC (methanol-ethyl acetate [1:5]) R_{f}=0.60; ¹H NMR (dimethyl-d₆ sulfoxide) δ 7.63 (br s), 7.31 (5H, m), 7.21 (4H, d, J=9.3 Hz), 6.87 (4H, d, J=9.3 Hz), 3.94 (1H, m), 3.71 (6H, s), 3.08-2.81 (4H, 2×m), 0.77 (9H, s), 0.00 (3H, s), −0.10 (3H, s); mass spectrum (El mode) m/z 508 [M+H]⁺.
4. (R)-1-(3-(bis(4-methoxyphenyl)(phenyl)methoxy)-2-(tert-butyldimethylsilyloxy)propyl)-3-(6-hydroxyhexyl)urea (MW 650.92) TLC (ethyl acetate) R_{f}=0.54; ¹H NMR (dimethyl-de sulfoxide) δ 7.38-7.16 (9H, m&d, J=9.0 Hz for the doublet), 6.84 (4H, d, J=9.0 Hz), 5.88 (1H, t, J=6.0 Hz), 5.52 (1H, t, J=6.0 Hz), 4.29 (1H, t, J=3.0 Hz), 3.79 (1H, m), 3.70 (6H, s), 3.34 (2H, m), 3.08 (2H, m), 2.90 (4H, m), 1.27 (8H, m), 0.80 (9H, s), 0.00 (3H, s), −0.05 (3H, s); mass spectrum (El mode) m/z 651 [M+H]⁺.
5. (R)-5-((bis(4-methoxyphenyl)(phenyl)methoxy)methyl)-2,2,3,3-tetramethyl-8-oxo-4-oxa-7,9-diaza-3-silapentadecan-15-yl 2-cyanoethyl diisopropylphosphoramidite (ULP3) (MW 851.14) ¹H NMR (dimethyl-de sulfoxide) δ 7.37 (2H, d, J=6.0 Hz), 7.22 (7H, d&m, J=9.0 Hz for the doublet), 6.84 (4H, d, J=9.0 Hz), 5.88 (1H, t, J=6.0 Hz), 5.22 (1H, t, J=6.0 Hz), 3.79 (1H, m), 3.74-3.64 (8H, m&s), 3.54 (4H, m), 3.07 (2H, m), 2.89 (4H, m), 2.72 (2H, t, J=6.0 Hz), 1.49 (2H, m), 1.26 (6H, m), 1.11 (12H, m), 0.80 (9H, s), 0.00 (3H, s), −0.05 (3H, s); ³¹P NMR (acetonitrile-d₃) δ 146.98; mass spectrum (El mode) m/z 851 [M+H]⁺.

### Example 6: Preparation of DMT-CPG

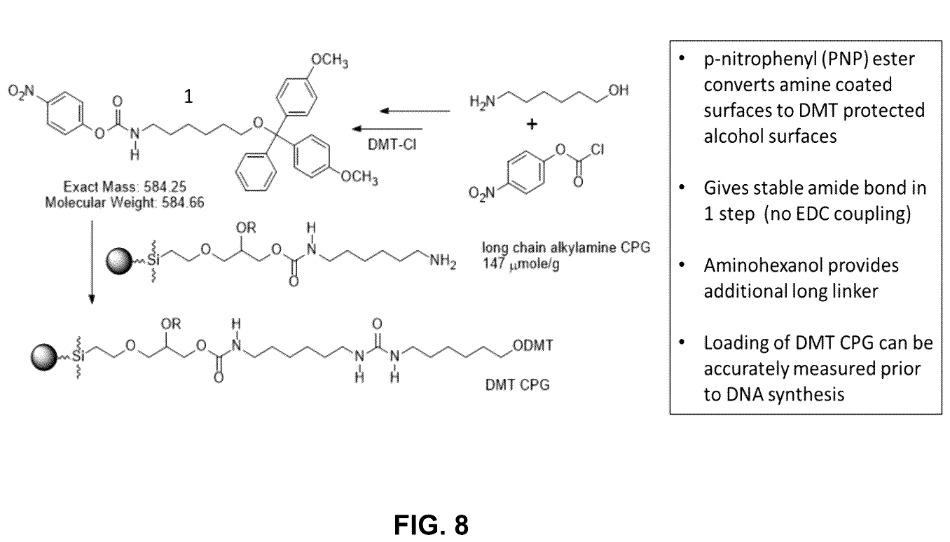

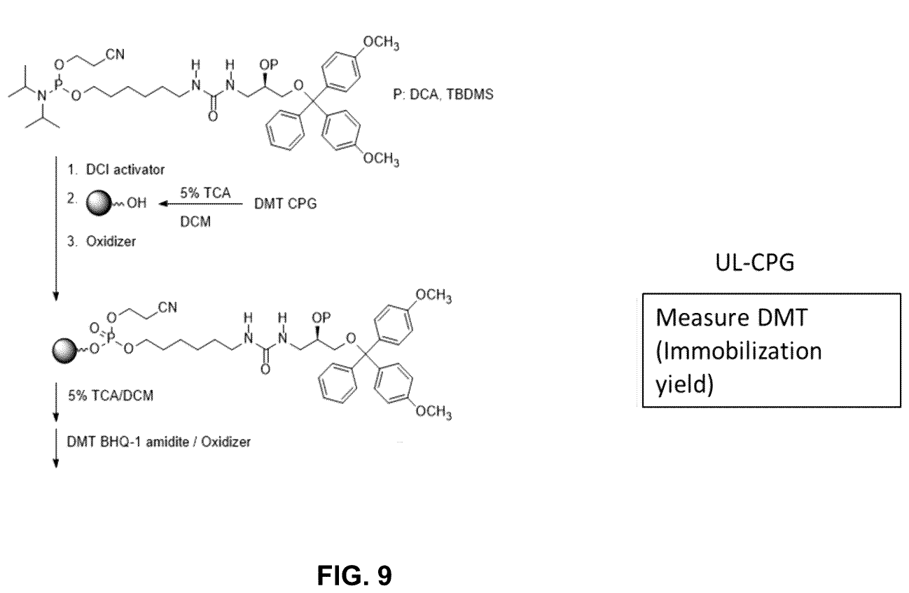

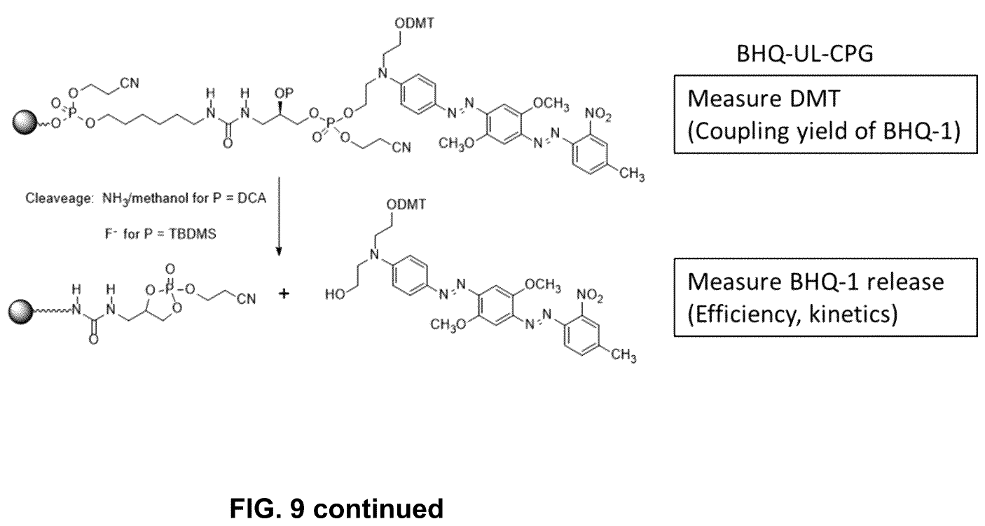

Performance of the Universal Linker Phosphoramidites were evaluated using controlled pore glass (CPG) solid supports. First, a model support (DMT-CPG) was prepared as shown in FIG. 8. Long chain alkylamine CPG (LCAA-CPG) was commercially obtained and modified to provide a solid surface with known loading of dimethoxytrityl groups per gram of CPG. A novel reagent was first prepared by reaction of p-nitrophenyl chloroformate with 6-aminohexanol. After the amine reacts specifically with the chloroformate (as evidenced by TLC) DMT-chloride is added in the same pot to react with the primary alcohol. The resulting DMT protected PNP carbamate was obtained as a pure product after silica gel chromatography. Further reaction with LCAA-CPG (147 μmole/g) gave DMT-CPG (99 μmole/g) and this was used for further assays of the ULP as shown in FIG. 9.

### Example 7: DMT-CPG Assay to Evaluate Immobilization, Coupling and Cleavage of ULP Reagents

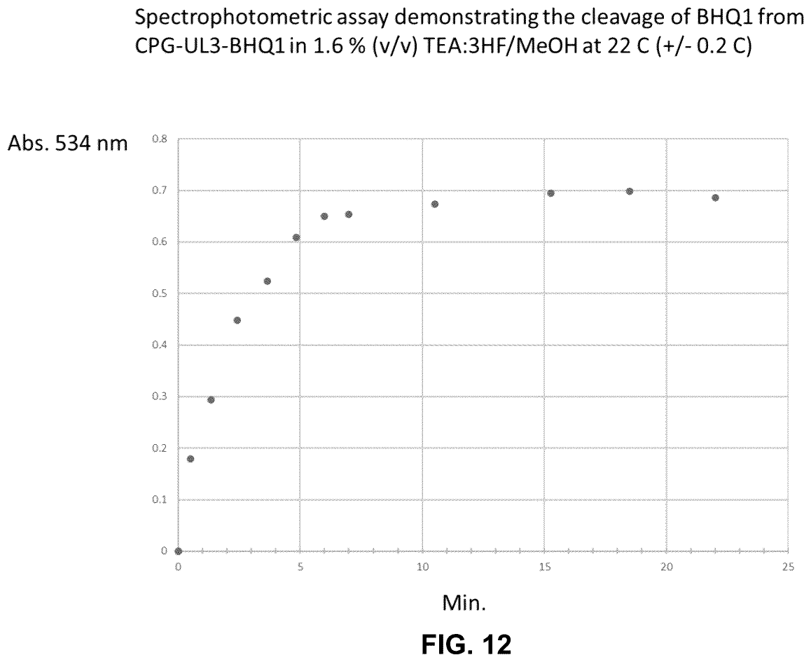

Behavior of the Universal Linker Phosphoramidites under different cleavage conditions was evaluated using DMT-CPG as shown in FIG. 9 for ULP1 and ULP3. For example, 3 μmoles (30 mg) of CPG (DMT loading=99 μmole/g) was weighed out and placed in a flow through DNA synthesis column with disposable frits (Biosearch Technologies, part number CL-1501-10). The DMT group was removed with 5% TCA/DCM (5 min at RT). The trityl cation concentration was recorded from visible spectrum peak at the absorbance maximum (˜498 nm) using extinction coefficient of 76 mL cm⁻¹ μmole⁻¹. The CPG was rinsed with acetonitrile and then coupled with 1 mL of a 1:1 mix of 0.1M ULP and 0.1M DCI (dicyanoimidazole) in acetonitrile. After 5 min, the ULP was washed off the CPG with acetonitrile, then oxidized with iodine in pyridine/water (5 min). The CPG was treated with 5% TCA/DCM and the trityl cation concentration was measured, and % coupling of the ULP to CPG was calculated. The UL-CPG was rinsed with acetonitrile and then coupled with 1 mL of a 1:1 mix of 0.1M BHQ-1 (Black Hole Quencher 1) DMT phosphoramidite (Biosearch Technologies, part number BNS-5051-50) and 0.1M DCI (4,5-dicyanoimidazole). After 5 min, the BHQ-1 amidite was washed off the CPG with acetonitrile, then oxidized with iodine in pyridine/water (5 min). UL-BHQ-1 CPG was treated with 5% TCA/DCM, the trityl cation concentration was measured, and % coupling of BHQ-1 to UL-CPG was calculated. The UL-BHQ-CPG was dried in vacuo (˜30 mg) and used to study release of BHQ-1 into solution. BHQ-1 concentration was recorded from visible spectrum peak at the absorbance maximum (˜534 nm) using extinction coefficient of 34 mL cm⁻¹ μmole⁻¹. As described in Example 1, 2-3 mg of UL1-BHQ-CPG or UL2-BHQ-CPG was treated with 4 M ammonia in anhydrous methanol and the % BHQ-1 release was measured over time. In a similar manner, UL3-BHQ-CPG was treated with 1.6% (v/v) TEA:3HF in anhydrous methanol and the % BHQ-1 release was measured over time. FIG. 12 shows the cleavage kinetics of BHQ1 from CPG-UL3-BHQ1 in 1.6% (v/v) TEA:3HF/MeOH at 22 C (+/−0.2 C) as determined by spectrophotometric assay.

### Example 8: Synthesis of DNA Primer Pairs Simultaneously with Universal Phosphoramidites

A common application for automated DNA synthesis is for production of PCR primers. PCR uses a pair of custom primers to direct DNA elongation toward each-other at opposite ends of the sequence being amplified. These primers are typically between 18 and 24 bases in length and must code for only the specific upstream and downstream sites of the sequence being amplified as shown in FIG. 10.

High concentration of PCR primers is required, preventing them from being synthesized on array instruments. But standard DNA synthesis of primers would benefit from a novel method to use Universal Linker phosphoramidites to make 2 primers in a single synthesis column. The UL phosphoramidite is introduced as a spacer between the Primer 1 and Primer 2 DNA sequences as shown in FIG. 11. After “trityl-off” DNA synthesis, the solid support is treated with 4M methylamine/MeOH. Primer 1 sequence is released from the solid support as usual with 3′-hydroxy group. The universal linker spacer is simultaneously cleaved to give Primer 2 sequence with a 3′-hydroxy group. Removal of protecting groups with AMA generates a 1:1 mixture of Primer 1 and Primer 2. Evaporation of AMA leaves a 1:1 mix of Primers, along with contaminating failure sequences and removed protecting groups. AMA requires use of Acetyl protected dC (Ac-dC) instead of Benzoyl protected dC to prevent transamidation by aqueous methylamine. The oligos are deprotected using concentrated ammonia (55° C., 1 hour) or AMA (55° C., 10 minutes).

Usually PCR primers are purified by removing the 5′-trityl group on the oligo and simply “desalting” using a gel filtration column. Gel filtration is the separation of the components of a mixture on the basis of molecular size and is the simplest form of chromatography for oligonucleotide purification. Cleaved protecting groups and short truncated sequences are retained in the gel matrix while larger oligonucleotide molecules elute quickly through the gel filtration column. Since Primer 1 and Primer 2 are about the same length, they elute in the same fraction. Concentration of the 1:1 mix of primers is determined by 260 nm absorbance using the combined extinction coefficients of the 2 oligos. The end user can use the 1:1 mixture directly in PCR without having to separately determine the concentration of each primer and calculating the volume of each.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A solid support system for synthesis of oligonucleotides, wherein the support comprises a planar surface and a universal linker, wherein the universal linker is coupled to the planar surface, wherein the planar surface comprises silicon, titanium, or platinum.

2. The solid support system of claim 1, wherein the planar surface is coated with an amine or carboxylic acid prior to attaching the universal linker.

3-4. (canceled)

5. The solid support system of claim 1, comprising Formula (I), (III), or (IV), optionally, wherein the universal linker is coupled to the planar surface by reacting the planar surface with a compound of Formula (II), (V), (VI), (VII), (VIII), (IX), (X), or combinations thereof, 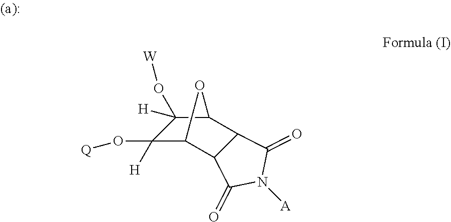
wherein, when A is a linking moiety attached to a coated platinum electrode comprising a substituted or unsubstituted aliphatic group, a substituted or unsubstituted aliphatic ether, a substituted or unsubstituted heteroalkyl group, a substituted or unsubstituted aromatic group, a substituted or unsubstituted heterocyclic group, one of W or Q is a blocking group that is cleavable under basic or neutral conditions, while the other of W or Q is H, or a blocking group that is cleavable under acidic conditions; or
wherein, when A is H, a substituted or unsubstituted aliphatic group, a substituted or unsubstituted aliphatic ether, a substituted or unsubstituted heteroalkyl group, a substituted or unsubstituted aromatic group, a substituted or unsubstituted heterocyclic group, one of Q or W is a linker moiety attached to a coated platinum electrode that is cleavable under basic or neutral conditions, while the other of W or Q is H, or a blocking group that is cleavable under acidic conditions; 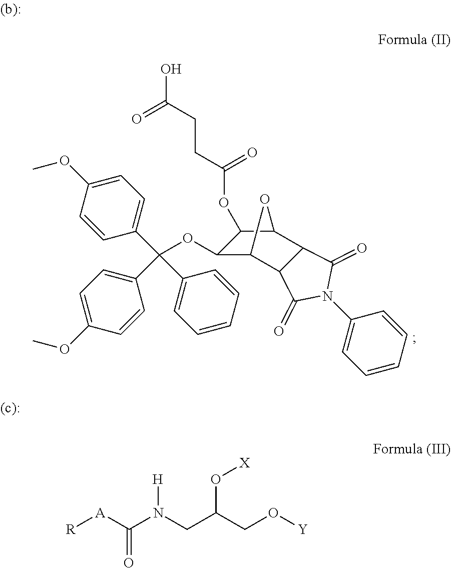
wherein
R is alkyl, aryl, heteroalkyl or heteroaryl attached to platinum electrode or other base material;
A is NH, O, S, alkyl, or aryl;
X is acyl, aroyl, or silyl; and
Y is dimethoxytrityl group or a protecting group removable under acidic or neutral conditions; 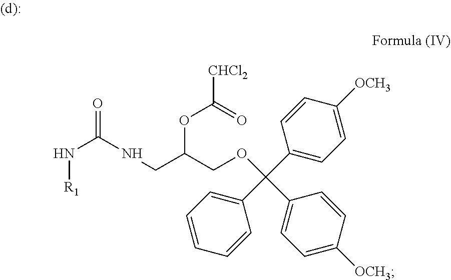
R₁ is alkyl, aryl, heteroalkyl, or heteroaryl attached to platinum electrode or other base material 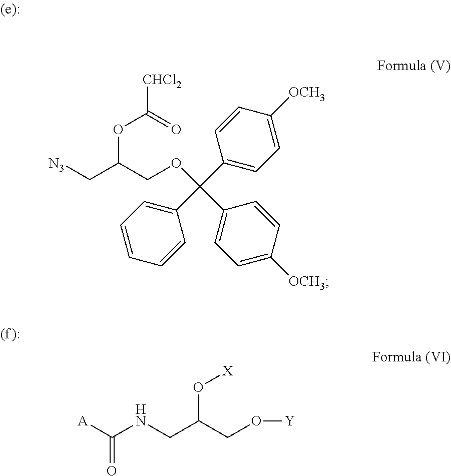
wherein A is 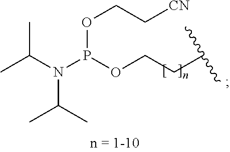
X is acyl, aroyl, or silyl, and
Y is dimethoxytrityl group or a protecting group removable under acidic or neutral conditions; 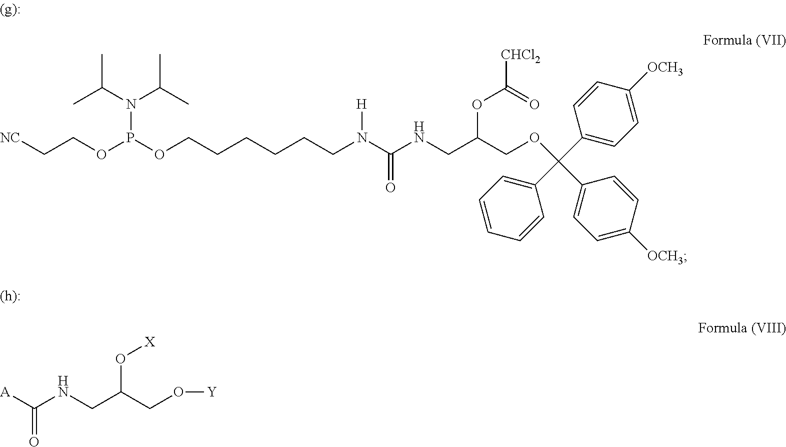
wherein A is 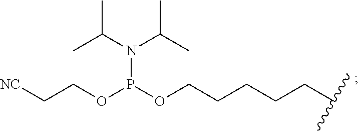 [SMILES: *CCCCCOP(OCCC#N)N(C(C)C)C(C)C]
X is acyl, aroyl, or silyl; and
Y is dimethoxytrityl group or a protecting group removable under acidic or neutral conditions;

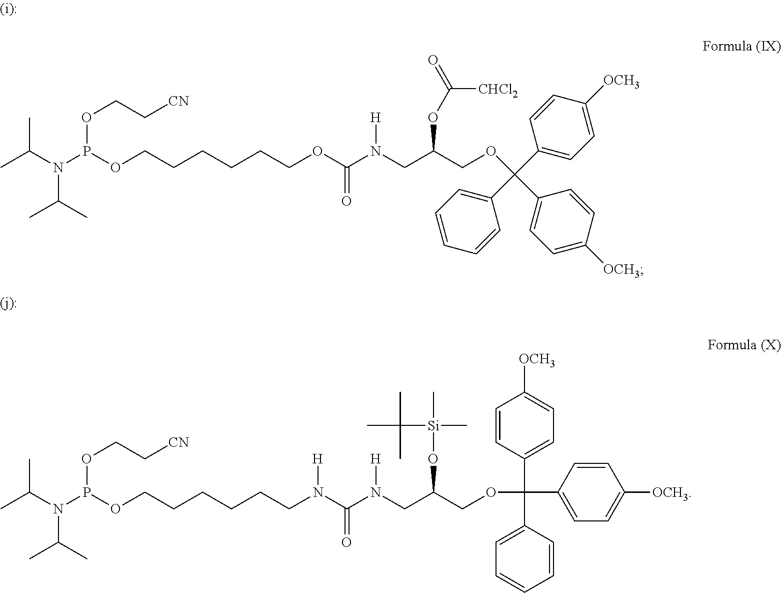

6. The solid support system of claim 5, wherein the compound is of Formula (II), (IV), (V), (VII), (IX), (X), or combinations thereof.

7. The solid support system of claim 6, wherein the compound is of Formula (VII), (IX), or (X).

8. The solid support system of claim 1, wherein the planar surface is coated with a monosaccharide or a disaccharide.

9. (canceled)

10. A method for synthesis of oligonucleotides comprising:
(a) providing an electrode device with a planar surface,
(b) depositing carboxylic acid electrochemically reducing the carboxylic acid onto the planar surface,
(c) depositing an amine coating onto the deposited carboxylic acid, wherein the planar surface comprises silicon titanium. or platinum;
(d) coupling the surface with a universal linker; and
(e) synthesizing the oligonucleotide.

11-13. (canceled)

14. The method of claim 10, wherein the electrode device comprises Formula (I), (III), or (IV), optionally, wherein the universal linker is coupled to the planar surface by reacting the planar surface with a compound of Formula (II), (V), (VI), (VII), (VIII), (IX), (X), or combinations thereof, 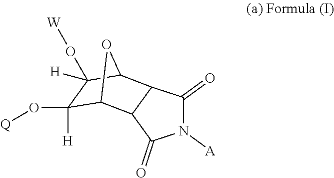
wherein, when A is a linking moiety attached to a coated platinum electrode comprising a substituted or unsubstituted aliphatic group, a substituted or unsubstituted aliphatic ether, a substituted or unsubstituted heteroalkyl group, a substituted or unsubstituted aromatic group, a substituted or unsubstituted heterocyclic group, one of W or Q is a blocking group that is cleavable under basic or neutral conditions, while the other of W or Q is H, or a blocking group that is cleavable under acidic conditions; or
wherein, when A is H, a substituted or unsubstituted aliphatic group, a substituted or unsubstituted aliphatic ether, a substituted or unsubstituted heteroalkyl group, a substituted or unsubstituted aromatic group, a substituted or unsubstituted heterocyclic group, one of Q or W is a linker moiety attached to a coated platinum electrode that is cleavable under basic or neutral conditions, while the other of W or Q is H, or a blocking group that is cleavable under acidic conditions; 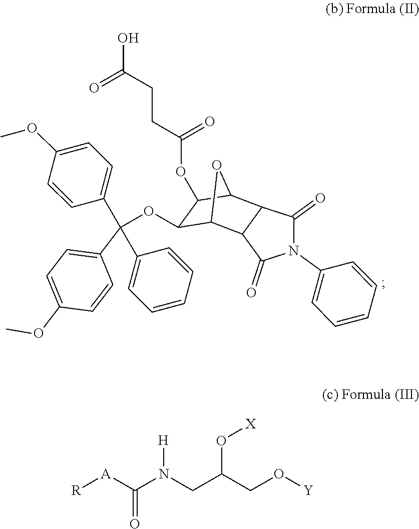
wherein
R is alkyl, aryl, heteroalkyl, or heteroaryl attached to platinum electrode or other base material;
A is NH, O, S, alkyl, or aryl;
X is acyl, aroyl, or silyl; and
Y is dimethoxytrityl group or a protecting group removable under acidic or neutral conditions; 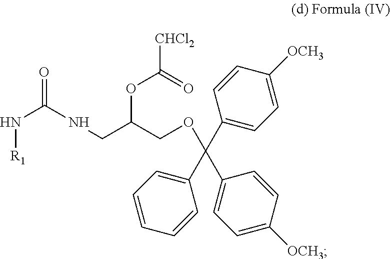
R₁ is alkyl, aryl, heteroalkyl, or heteroaryl attached to platinum electrode or other base material; 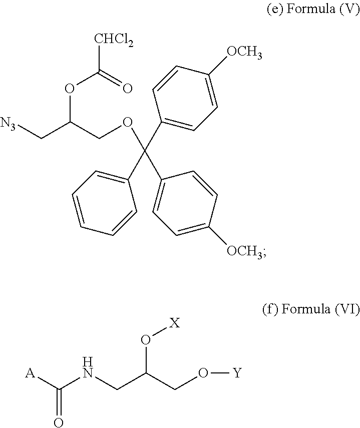
wherein
A is 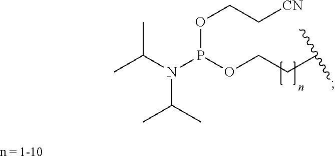
X is acyl, aroyl, or silyl; and
Y is dimethoxytrityl group or a protecting group removable under acidic or neutral conditions; 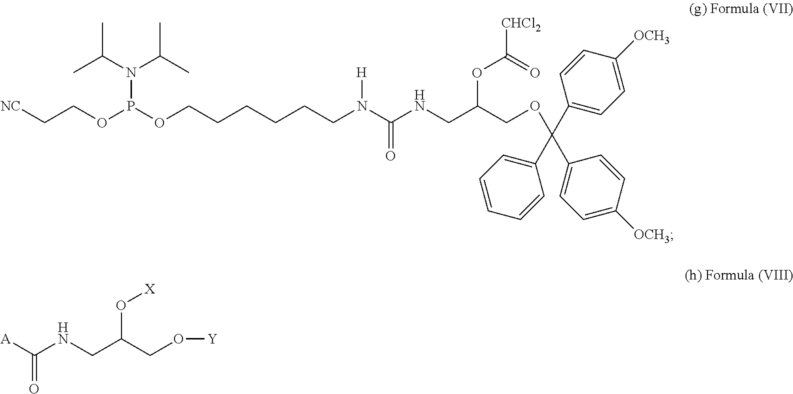
wherein
A is 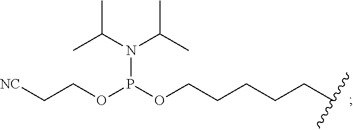 [SMILES: *CCCCCOP(OCCC#N)N(C(C)C)C(C)C]
X is acyl, aroyl, or silyl; and
Y is dimethoxytrityl group or a protecting group removable under acidic or neutral conditions;

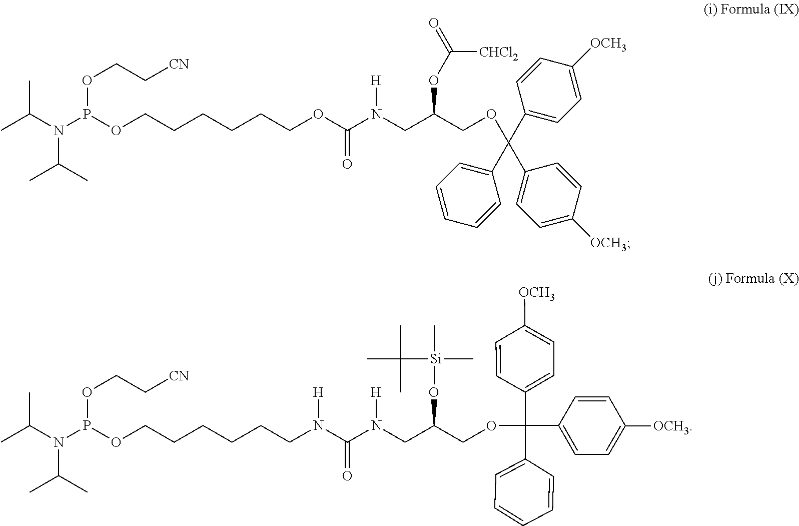

15. The method of claim 14, wherein the compound is selected from Formula (II), (IV), (V), (VII), (IX), (X), and a combination thereof.

16. The method of claim 15, wherein the compound is selected from (VII), (IX), and (X).

17. The method of claim 10, wherein the planar surface is coated with a monosaccharide or a disaccharide.

18. A method for synthesis of oligonucleotide primer pairs comprising
providing a solid support comprising a first universal linker immobilized on a surface of the solid support,
performing a first phosphoramidite DNA synthesis to generate a first oligonucleotide primer, wherein the 3′ end of the first oligonucleotide primer is attached to the first universal linker,
coupling a second universal linker to the 5′ end of the first oligonucleotide primer,
performing a second phosphoramidite DNA synthesis to generate a second oligonucleotide primer, wherein the 3′ end of the second oligonucleotide primer is attached to the second universal linker, and
contacting the solid support with a releasing agent thereby releasing the first and the second oligonucleotide primers from the solid support, wherein each of the released first oligonucleotide primer and the released second oligonucleotide primer comprises a 3′-hydroxy group,
wherein the first universal linker is immobilized to the solid support by reacting the solid support with a first compound of 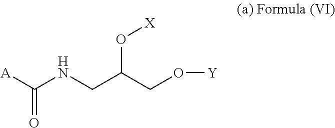
wherein
A is 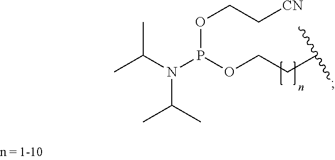
X is acyl, aroyl, or silyl; and
Y is dimethoxytrityl group or a protecting group removable under acidic or neutral conditions: 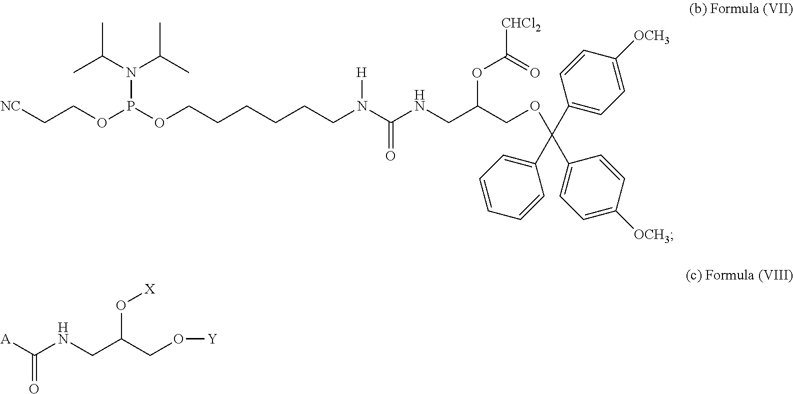
wherein
A is 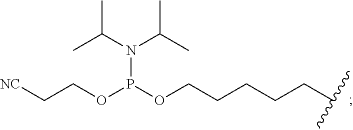 [SMILES: *CCCCCOP(OCCC#N)N(C(C)C)C(C)C]
X is acyl, aroyl, or silyl; and
Y is dimethoxytrityl group or a protecting group removable under acidic or neutral conditions; 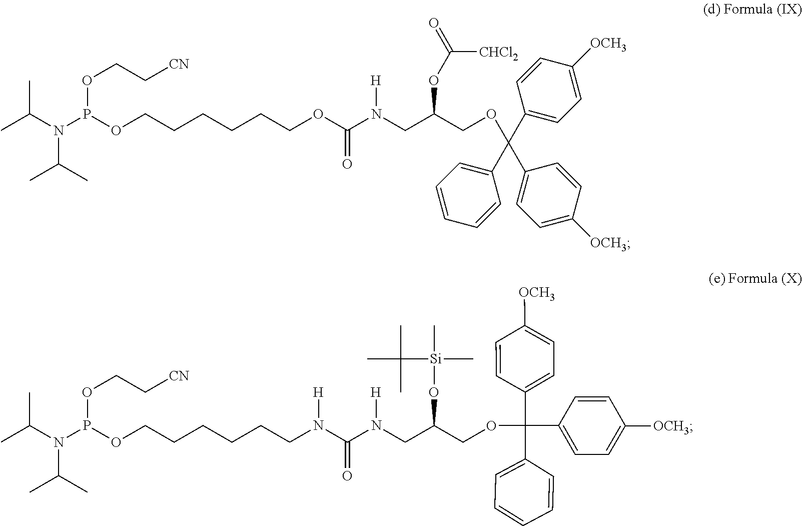
or combinations thereof.

19. (canceled)

20. The method of claim 18, wherein the first compound is Formula (VII), (IX), or (X).

21. The method of claim 18, wherein the second universal linker is attached to the first oligonucleotide primer by reacting the first oligonucleotide primer with a second compound of Formula (VII), (IX), or (X).

22-28. (canceled)

29. A compound of Formula (XI), 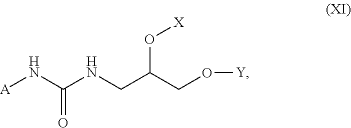
wherein
A is 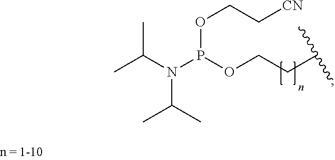
X is acyl, aroyl, or silyl, and
Y is dimethoxytrityl group or a protecting group removable under acidic or neutral conditions.

30. The compound of claim 29, wherein n is 5.

31. The compound of claim 29, wherein X is silyl.

32. The compound of claim 29, wherein the silyl is trimethylsilyl, Triethylsilyl, tert-butyldiphenylsilyl, tert-butyldimethylsilyl, or triisopropylsilyl.

33. The compound of claim 32, wherein the silyl is tert-butyldimethylsilyl.

34-42. (canceled)

43. The solid support system of claim 1, wherein the support is a complementary metal-oxide semiconductor (CMOS).
